(19) United States
(12) Patent Application Publication
(10) Pub. No.: US 2008/0065154 A1
(43) Pub. Date: Mar. 13, 2008

(54) Surgical staple

(75) Inventors: Randall N. Allard, Germantown, TN (US); Larry T. McBride, Ripley, TN (US); Lehmann K. Li, Milford, CT (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: WARSAW ORTHOPEDIC, INC, Warsaw, IN (US)

(21) Appl. No.: 11/518,069

(22) Filed: Sep. 8, 2006

(51) Int. Cl. A61B 17/08

(52) U.S. Cl. 606/219

(57) ABSTRACT

A surgical staple is disclosed and can include a base and at least one tine that can extend from the base. The at least one tine includes an enlarged portion to provide a greater surface area to engage tissue when installed in a patient.

### FIELD OF THE DISCLOSURE

The present disclosure relates generally to surgical staples. More specifically, the present disclosure relates to surgical staples having various features configured to engage bone, flesh, or a combination thereof.

### BACKGROUND

Surgical staples can be used to close surgical wounds and other wounds. Further, surgical staples can be used to treat certain spinal deformities, such as scoliosis. Some surgical staples can be made from shape memory metal alloys. A surgical staple made from a shape memory metal alloy can be deformed from an original shape and installed in a patient. Then, heat can be applied to the surgical staple to return the deformed staple to the original shape.

### BRIEF DESCRIPTION OF THE DRAWINGS

FIG. 1 is a perspective view of a first embodiment of a two-tine surgical staple;

FIG. 2 is a plan view of the first embodiment of the two-tine surgical staple in an open configuration;

FIG. 3 is a plan view of the first embodiment of the two-tine surgical staple in a closed configuration;

FIG. 4 is a perspective view of a second embodiment of a two-tine surgical staple;

FIG. 5 is a plan view of the second embodiment of the two-tine surgical staple in a closed configuration;

FIG. 6 is a plan view of the second embodiment of the two-tine surgical staple in an open configuration;

FIG. 7 is a plan view of a third embodiment of the two-tine surgical staple with the first tine and the second tine in a straight configuration;

FIG. 8 is a perspective view of the third embodiment of a two-tine surgical staple with a first tine and a second tine in a straight configuration;

FIG. 9 is a perspective view of the third embodiment of the two-tine surgical staple with the first tine and the second tine in a twisted configuration;

FIG. 10 is a perspective view of a fourth embodiment of a two-tine surgical staple with a first tine and a second tine in a straight configuration;

FIG. 11 is a perspective view of the fourth embodiment of the two-tine surgical staple with the first tine and the second tine in a curved configuration;

FIG. 12 is a plan view of the fourth embodiment of the two-tine surgical staple installed within a superior vertebra and an inferior vertebra with the first tine and the second tine in the straight configuration;

FIG. 13 is a plan view of the fourth embodiment of the two-tine surgical staple installed within a superior vertebra and an inferior vertebra with the first tine and the second tine in the curved configuration;

FIG. 14 is a first plan view of a fifth embodiment of a two-tine surgical staple;

FIG. 15 is a second plan view of the fifth embodiment of the two-tine surgical staple;

FIG. 16 is a third plan view of the fifth embodiment of the two-tine surgical staple;

FIG. 17 is a first plan view of a sixth embodiment of a two-tine surgical staple;

FIG. 18 is a second plan view of the sixth embodiment of the two-tine surgical staple;

FIG. 19 is a third plan view of the sixth embodiment of the two-tine surgical staple;

FIG. 20 is a plan view of a seventh embodiment of a two-tine surgical staple;

FIG. 21 is a second plan view of the seventh embodiment of the two-tine surgical staple;

FIG. 22 is a third plan view of the seventh embodiment of the two-tine surgical staple;

FIG. 23 is a plan view of a eighth embodiment of a two-tine surgical staple;

FIG. 24 is a second plan view of the eighth embodiment of the two-tine surgical staple;

FIG. 25 is a third plan view of the eighth embodiment of the two-tine surgical staple;

FIG. 26 is a plan view of a ninth embodiment of a two-tine surgical staple;

FIG. 27 is a second plan view of the ninth embodiment of the two-tine surgical staple;

FIG. 28 is a third plan view of the ninth embodiment of the two-tine surgical staple;

FIG. 29 is a plan view of a tenth embodiment of a two-tine surgical staple;

FIG. 30 is a second plan view of the tenth embodiment of the two-tine surgical staple;

FIG. 31 is a third plan view of the tenth embodiment of the two-tine surgical staple;

FIG. 32 is a plan view of a eleventh embodiment of a two-tine surgical staple;

FIG. 33 is a second plan view of the eleventh embodiment of the two-tine surgical staple;

FIG. 34 is a third plan view of the eleventh embodiment of the two-tine surgical staple;

FIG. 35 is a perspective view of a twelfth embodiment of a two-tine surgical staple;

FIG. 36 is a first plan view of the twelfth embodiment of the two-tine surgical staple;

FIG. 37 is a second plan view of the twelfth embodiment of the two-tine surgical staple;

FIG. 38 is a third plan view of the twelfth embodiment of the two-tine surgical staple;

FIG. 39 is a fourth plan view of the twelfth embodiment of the two-tine surgical staple;

FIG. 40 is a fifth plan view of the twelfth embodiment of the two-tine surgical staple;

FIG. 41 is a perspective view of a first embodiment of a three-tine surgical staple;

FIG. 42 is a first plan view of the first embodiment of the three-tine surgical staple;

FIG. 43 is a second plan view of the first embodiment of the three-tine surgical staple;

FIG. 44 is a third plan view of the first embodiment of the three-tine surgical staple;

FIG. 45 is a fourth plan view of the first embodiment of the three-tine surgical staple;

FIG. 46 is a fifth plan view of the first embodiment of the three-tine surgical staple;

FIG. 47 is a sixth plan view of the first embodiment of the three-tine surgical staple;

FIG. 48 is a perspective view of a second embodiment of a three-tine surgical staple;

FIG. 49 is a first plan view of the second embodiment of the three-tine surgical staple;

FIG. 50 is a second plan view of the second embodiment of the three-tine surgical staple;

FIG. 51 is a third plan view of the second embodiment of the three-tine surgical staple;

FIG. 52 is a fourth plan view of the second embodiment of the three-tine surgical staple;

FIG. 53 is a fifth plan view of the second embodiment of the three-tine surgical staple;

FIG. 54 is a sixth plan view of the second embodiment of the three-tine surgical staple;

FIG. 55 is a perspective view of a first embodiment of a four-tine surgical staple;

FIG. 56 is a first plan view of the first embodiment of the four-tine surgical staple in a closed configuration;

FIG. 57 is a second plan view of the first embodiment of the four-tine surgical staple in a closed configuration;

FIG. 58 is a third plan view of the first embodiment of the four-tine surgical staple in an open configuration;

FIG. 59 is a perspective view of a second embodiment of a four-tine surgical staple;

FIG. 60 is a first plan view of the second embodiment of the four-tine surgical staple;

FIG. 61 is a second plan view of the second embodiment of the four-tine surgical staple;

FIG. 62 is a third plan view of the second embodiment of the four-tine surgical staple;

FIG. 63 is a fourth plan view of the second embodiment of the four-tine surgical staple;

FIG. 64 is a fifth plan view of the second embodiment of the four-tine surgical staple;

FIG. 65 is a sixth plan view of the second embodiment of the four-tine surgical staple;

FIG. 66 is a perspective view of a third embodiment of a four-tine surgical staple;

FIG. 67 is a first plan view of the third embodiment of the four-tine surgical staple;

FIG. 68 is a second plan view of the third embodiment of the four-tine surgical staple;

FIG. 69 is a third plan view of the third embodiment of the four-tine surgical staple;

FIG. 70 is a fourth plan view of the third embodiment of the four-tine surgical staple; and

FIG. 71 is a fifth plan view of the third embodiment of the four-tine surgical staple.

### DETAILED DESCRIPTION OF THE DRAWINGS

A surgical staple is disclosed and can include a base and at least one tine that can extend from the base. The at least one tine includes an enlarged portion to provide a greater surface area to engage tissue when installed in a patient.

In another embodiment, a surgical staple is disclosed and can include a base. A first tine can extend from the base. The first tine can include an enlarged portion formed along the first tine. Further, a second tine can extend from the base opposite the first tine. The second tine can include an enlarged portion formed along the second tine. The surgical staple can be moved between a closed configuration and an open configuration.

In yet another embodiment, a surgical staple is disclosed and can include a base. A first tine can extend from the base. The first tine can include an enlarged portion formed along the first tine. Further, a second tine can extend from the base opposite the first tine. The second tine can include an enlarged portion formed along the second tine. Also, the surgical staple can be moved between a straight configuration and a twisted configuration.

In still another embodiment, a surgical staple is disclosed and can include a base. A first tine can extend from the base. The first tine can include an enlarged portion formed along the first tine. Further, a second tine can extend from the base adjacent to the first tine. The second tine can include an enlarged portion formed along the second tine. A third tine can extend from the base opposite the first tine and the second tine. The third tine can include an enlarged portion formed along the third tine. Moreover, the surgical staple can be moved between a closed configuration and an open configuration.

In still yet another embodiment, a surgical staple is disclosed and can include a base. A first tine can extend from the base. The first tine can include an enlarged portion formed along the first tine. Further, a second tine can extend from the base adjacent to the first tine. The second tine can include an enlarged portion formed along the second tine. A third tine can extend from the base opposite the first tine and the second tine. The third tine can include an enlarged portion formed along the third tine. The surgical staple can be moved between a straight configuration and a twisted configuration.

In another embodiment, a surgical staple is disclosed and can include a base. A first tine can extend from the base. The first tine can include an enlarged portion formed along the first tine. Further, a second tine can extend from the base adjacent to the first tine. The second tine can include an enlarged portion formed along the second tine. A third tine can extend from the base opposite the first tine and the second tine. The third tine can include an enlarged portion formed along the third tine. A fourth tine can extend from the base adjacent to the third tine. The fourth tine can include an enlarged portion formed along the fourth tine. Additionally, the surgical staple can be moved between a closed configuration and an open configuration.

In yet another embodiment, a surgical staple is disclosed and can include a base. A first tine can extend from the base. The first tine can include an enlarged portion formed along the first tine. Further, a second tine can extend from the base adjacent to the first tine. The second tine can include an enlarged portion formed along the second tine. A third tine can extend from the base opposite the first tine and the second tine. The third tine can include an enlarged portion formed along the third tine. A fourth tine can extend from the base adjacent to the third tine. The fourth tine can include an enlarged portion formed along the fourth tine. Additionally, the surgical staple can be moved between a straight configuration and a twisted configuration.

In another embodiment, a surgical staple is disclosed and can include a generally trough-shaped base. A first hollow tine can extend from the base. Also, a second hollow tine can extend from the base opposite the first hollow tine.

### Description of a First Embodiment of a Two-Tine Surgical Staple

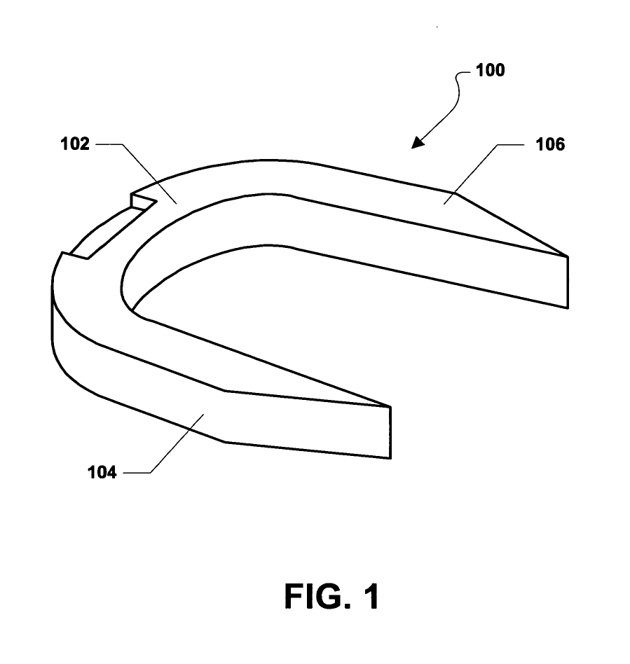

Referring initially to FIG. 1 through FIG. 3, a first embodiment of a two-tine surgical staple is shown and is generally designated 100. As shown, the two-tine surgical staple 100 can be generally U-shaped and can include a base 102. A first tine 104 and a second tine 106 can extend from the base 102.

As depicted, the first tine 104 can include a proximal end 110 and a distal end 112. Moreover, the first tine 104 can include an inner surface 114 and an outer surface 116. In a particular embodiment, the inner surface 114 of the first tine 104 can be relatively longer than the outer surface 116 of the first tine 104. As such, the distal end 112 of the first tine 104 can be angled from the inner surface 114 to the outer surface 116 and this angled portion can face outward relative to the surgical staple 100.

The second tine 106 can include a proximal end 120 and a distal end 122. Moreover, the second tine 106 can include an inner surface 124 and an outer surface 126. In a particular embodiment, the inner surface 124 of the second tine 106 can be relatively longer than the outer surface 126 of the second tine 106. As such, the distal end 122 of the second tine 106 can be angled from the inner surface 124 to the outer surface 126 and this angled portion can face outward relative to the surgical staple 100.

FIG. 1 through FIG. 3 also indicate that the base 102 of the surgical staple 100 can be formed with a slot 130. In a particular embodiment, the slot 130 can facilitate retrieval and insertion of the surgical staple 100.

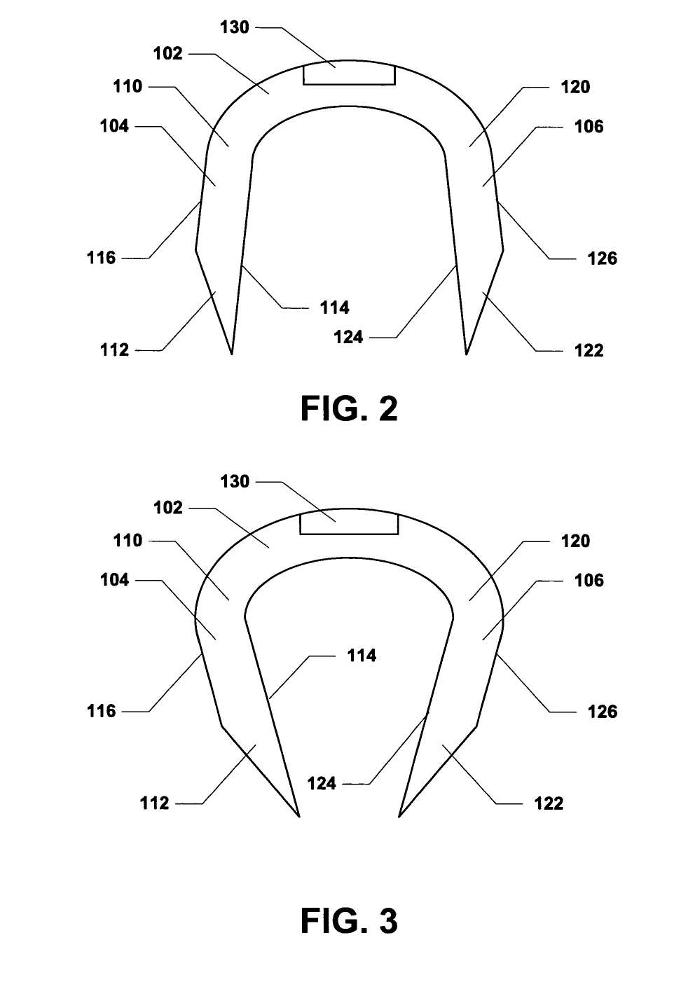

The surgical staple 100 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. As shown in FIG. 2 and FIG. 3, the surgical staple 100 can be moved between an open configuration, shown in FIG. 2, and a closed configuration, shown in FIG. 3. In the open configuration, the tines 104, 106 are angled outward relative to the base 102. In the closed configuration, the tines 104, 106 are angled inward relative to the base 102. Further, a distance between the tines 104, 106 in the closed configuration is less than a distance between the tines 104, 106 in the open configuration. In a particular embodiment, the surgical staple 100 can be moved from the open configuration to the closed configuration by applying heat to the surgical staple 100.

### Description of a Second Embodiment of a Two-Tine Surgical Staple

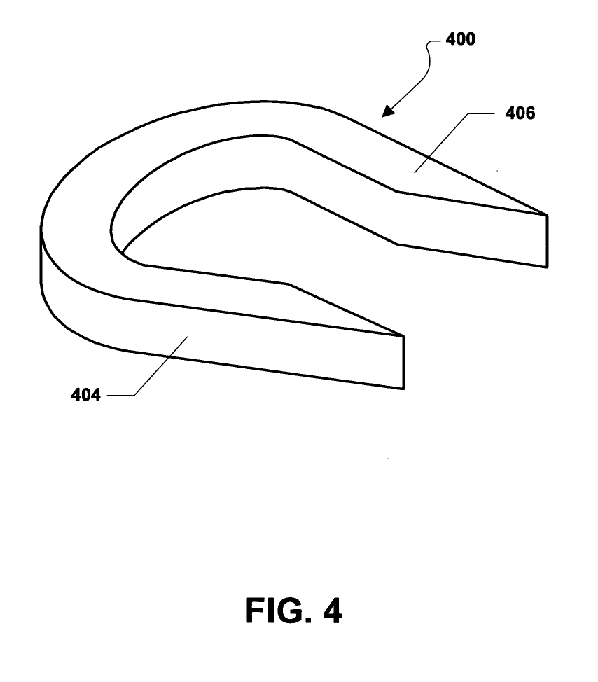

Referring to FIG. 4 through FIG. 6, a second embodiment of a two-tine surgical staple is shown and is generally designated 400. As shown, the two-tine surgical staple 400 can be generally U-shaped and can include a base 402. A first tine 404 and a second tine 406 can extend from the base 402.

As depicted, the first tine 404 can include a proximal end 410 and a distal end 412. Moreover, the first tine 404 can include an inner surface 414 and an outer surface 416. In a particular embodiment, the inner surface 414 of the first tine 404 can be relatively shorter than the outer surface 416 of the first tine 404. As such, the distal end 412 of the first tine 404 can be angled from the inner surface 414 to the outer surface 416 and this angled portion can face inward relative to the surgical staple 400.

The second tine 406 can include a proximal end 420 and a distal end 422. Moreover, the second tine 406 can include an inner surface 424 and an outer surface 426. In a particular embodiment, the inner surface 424 of the second tine 406 can be relatively shorter than the outer surface 426 of the second tine 406. As such, the distal end 422 of the second tine 406 can be angled from the inner surface 424 to the outer surface 426 and this angled portion can face inward relative to the surgical staple 400.

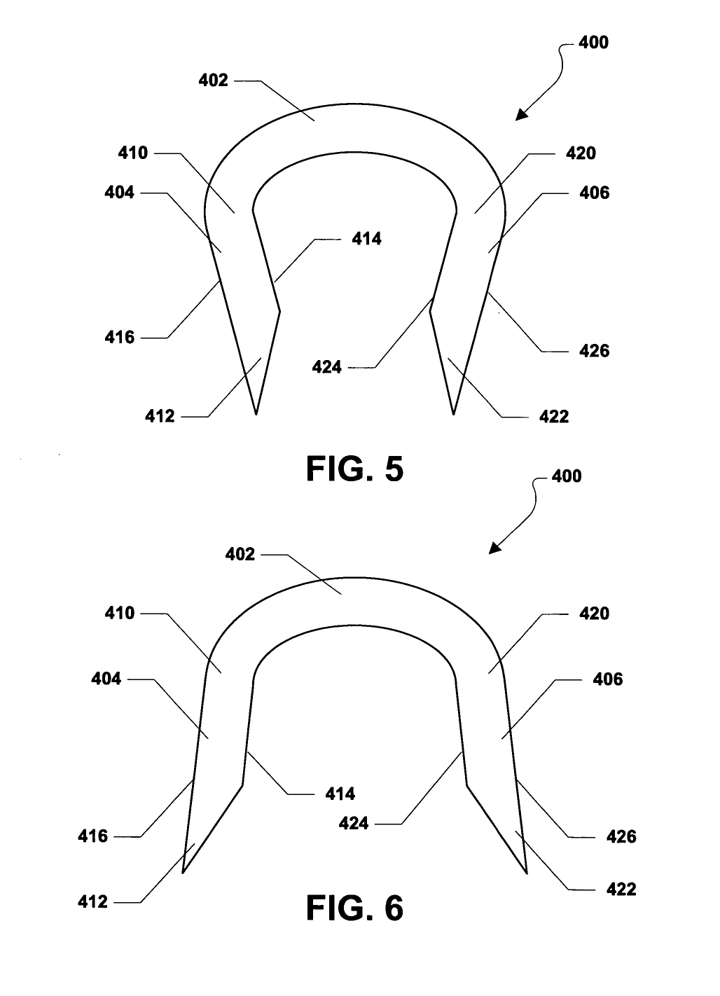

The surgical staple 400 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. As shown in FIG. 5 and FIG. 6, the surgical staple 400 can be moved between a closed configuration, shown in FIG. 5, and an open configuration, shown in FIG. 6. In a particular embodiment, the surgical staple 400 can be moved from the open configuration to the closed configuration by applying heat to the surgical staple 400.

### Description of a Third Embodiment of a Two-Tine Surgical Staple

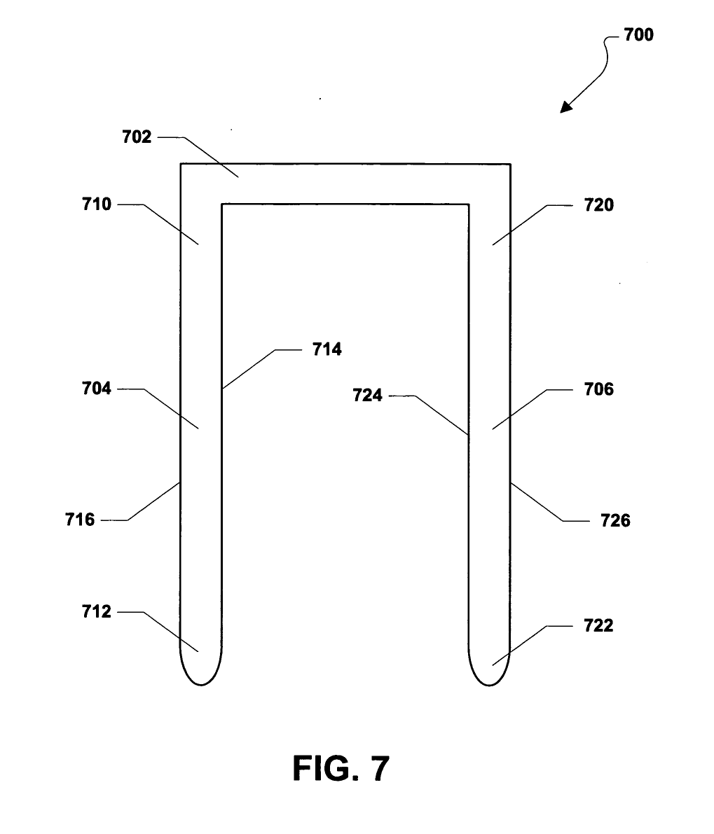

Referring to FIG. 7 through FIG. 9, a third embodiment of a two-tine surgical staple is shown and is generally designated 700. As shown, the two-tine surgical staple 700 can be generally U-shaped and can include a base 702. A first tine 704 and a second tine 706 can extend from the base 702.

As depicted, the first tine 704 can include a proximal end 710 and a distal end 712. Moreover, the first tine 704 can include an inner surface 714 and an outer surface 716. In a particular embodiment, the inner surface 714 of the first tine 704 can be substantially the same length as the outer surface 716 of the first tine 704.

The second tine 706 can include a proximal end 720 and a distal end 722. Moreover, the second tine 706 can include an inner surface 724 and an outer surface 726. In a particular embodiment, the inner surface 724 of the second tine 706 can be substantially the same length as the outer surface 726 of the second tine 706.

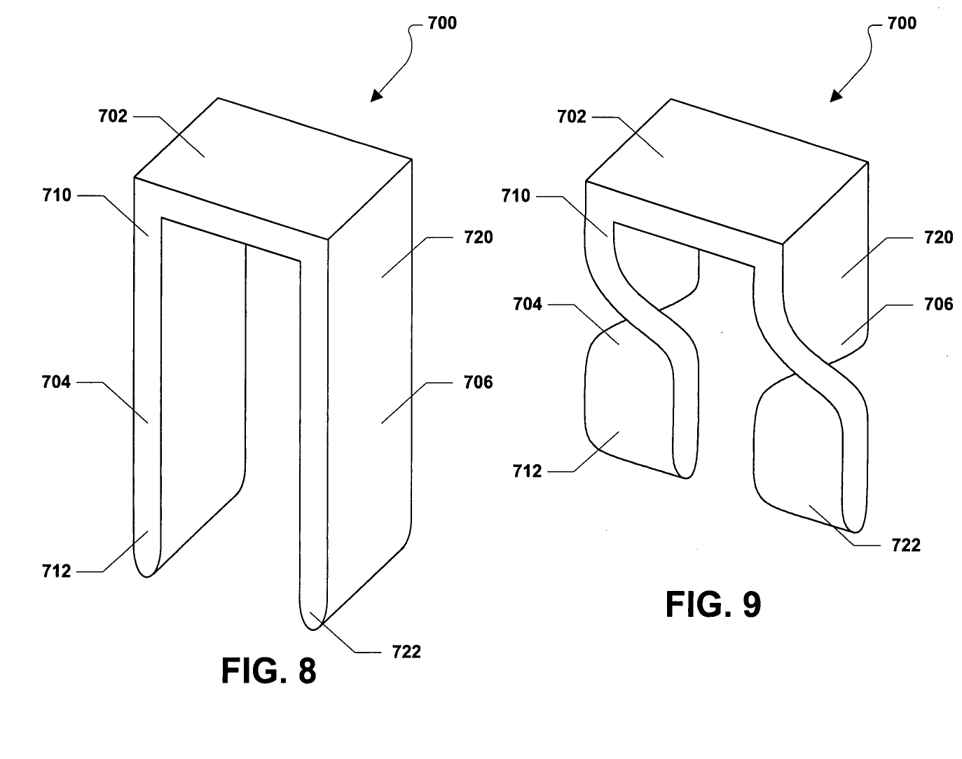

The surgical staple 700 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. In a particular embodiment, the surgical staple 700 can be moved between a straight configuration, shown in FIG. 7 and FIG. 8, and a twisted configuration, shown in FIG. 9.

In the straight configuration, the distal end 712 of the first tine 704 can be substantially aligned with, and substantially parallel to, the proximal end 710 of the first tine 704. Also, in the straight configuration, the distal end 722 of the second tine 704 can be substantially aligned with, and substantially parallel to, the proximal end 720 of the second tine 706.

Further, in the twisted configuration, the distal end 712 of the first tine 704 can be twisted relative to the proximal end 710 of the first tine 704. For example, the distal end 712 can be twisted ninety degrees (90°) relative to the proximal end 710. As such, the distal end 712 of the first tine 704 can be substantially perpendicular to the proximal end 710 of the first tine 704 in the twisted configuration. In the twisted configuration, the distal end 722 of the second tine 706 can be twisted relative to the proximal end 720 of the second tine 706. For example, the distal end 722 can be twisted ninety degrees (90°) relative to the proximal end 720. As such, the distal end 722 of the second tine 706 can be substantially perpendicular to the proximal end 720 of the second tine 706 in the twisted configuration.

In the twisted configuration, the tines 704, 706 can engage tissue, e.g., bone or flesh, and substantially minimize the likelihood that the surgical staple 700 will be prematurely withdrawn from the tissue.

In a particular embodiment, the tines 704, 706 of the surgical staple 700 can be moved from the straight configuration to the twisted configuration by applying heat to the surgical staple 700.

### Description of a Fourth Embodiment of a Two-Tine Surgical Staple

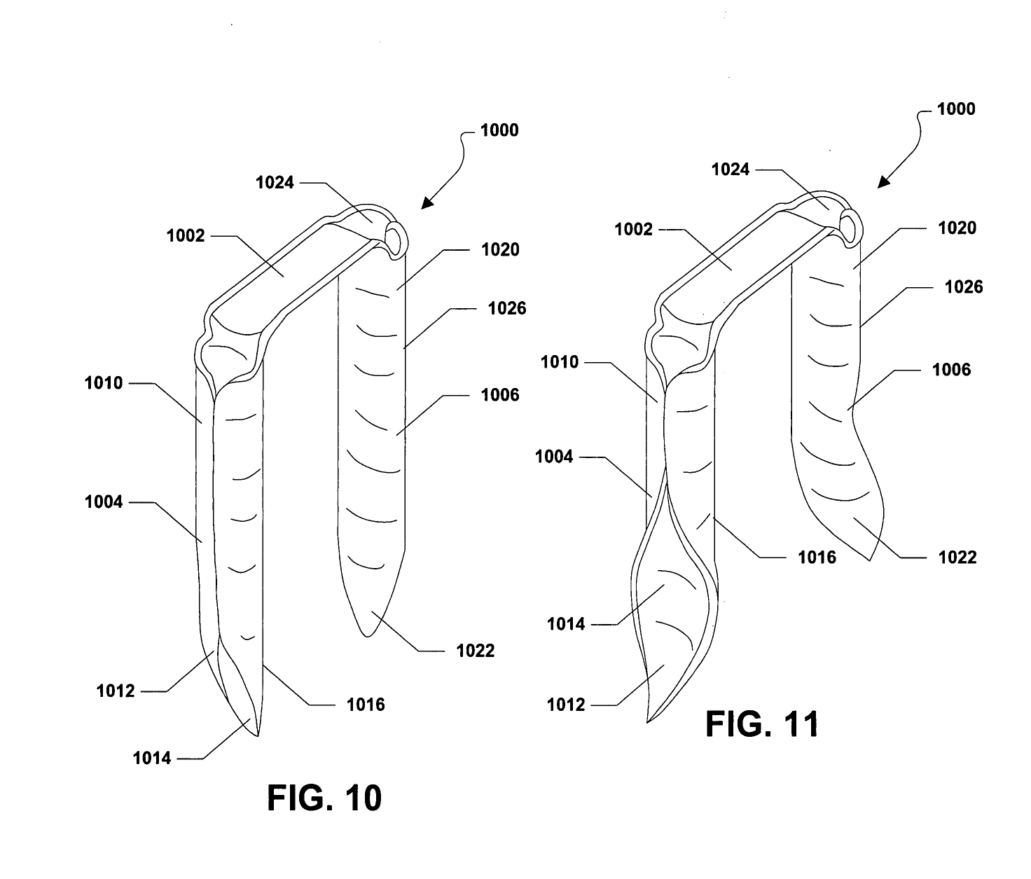

Referring to FIG. 10 through FIG. 13, a fourth embodiment of a two-tine surgical staple is shown and is generally designated 1000. As shown, the two-tine surgical staple 1000 can be generally U-shaped and can include a base 1002. A first tine 1004 and a second tine 1006 can extend from the base 1002. In a particular embodiment, the base 1002 can be generally trough-shaped. Further, the first tine 1004 and the second tine 1006 can be generally hollow and generally cylindrical.

As depicted, the first tine 1004 can include a proximal end 1010 and a distal end 1012. Moreover, the first tine 1004 can include an interior surface 1014 and an exterior surface 1016. The second tine 1006 can include a proximal end 1020 and a distal end 1022. Moreover, the second tine 1006 can include an interior surface 1024 and an exterior surface 1026.

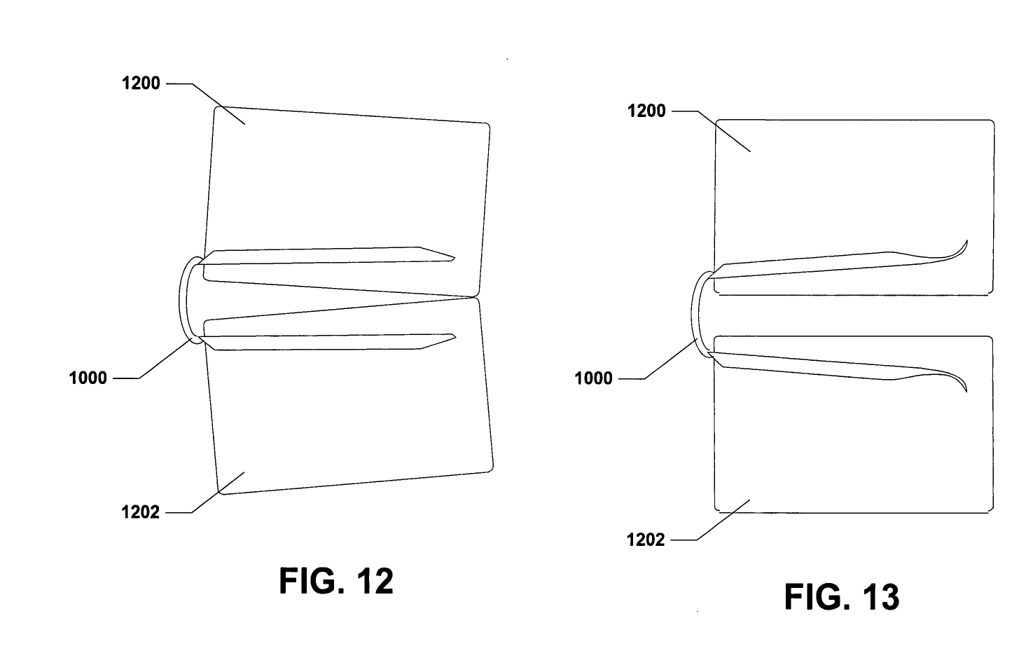

The surgical staple 1000 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. In a particular embodiment, the surgical staple 1000 can be moved between a closed configuration, shown in FIG. 10 and FIG. 12, and an open configuration, shown in FIG. 11 and FIG. 13.

In the closed configuration, the tines 1004, 1006 can be substantially straight and can extend substantially perpendicular from the base 1002 of the surgical stale 1000. In the open configuration, the tines 1004, 1006 can bend outward relative to each other. Further, the distal end 1012, 1022 of each tine 1004, 1006 can open up as each tine 1004, 1006 bends outward. In the open configuration the tines 1004, 1006, e.g., the distal ends 1012, 1022 thereof, can engage tissue, e.g., bone or flesh, and substantially minimize the likelihood that the surgical staple 1000 will be prematurely withdrawn from the tissue. Moreover, the hollow tines 1004, 1006 can provide greater surface area for engaging the tissue.

In a particular embodiment, the surgical staple 1000 can be moved from the closed configuration to the open configuration by applying heat to the surgical staple 1000.

As shown in FIG. 12, in the closed configuration, the surgical staple 1000 can installed, or otherwise inserted into adjacent vertebrae, e.g., into a superior vertebra 1200 and an inferior vertebra 1202. Thereafter, the surgical staple 1000 can be moved to the open configuration in order to distract the vertebrae 1200, 1200.

### Description of a Fifth Embodiment of a Two-Tine Surgical Staple

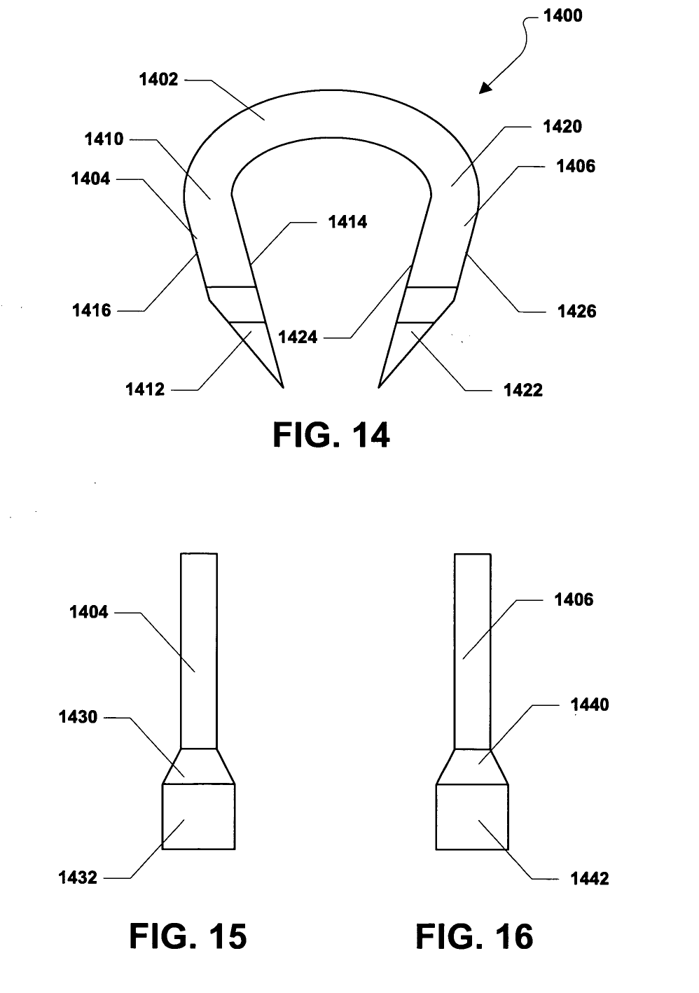

Referring to FIG. 14 through FIG. 16, a fifth embodiment of a two-tine surgical staple is shown and is generally designated 1400. As shown, the two-tine surgical staple 1400 can be generally U-shaped and can include a base 1402. A first tine 1404 and a second tine 1406 can extend from the base 1402.

As depicted, the first tine 1404 can include a proximal end 1410 and a distal end 1412. Moreover, the first tine 1404 can include an inner surface 1414 and an outer surface 1416. In a particular embodiment, the inner surface 1414 of the first tine 1404 can be relatively longer than the outer surface 1416 of the first tine 1404. As such, the distal end 1412 of the first tine 1404 can be angled from the inner surface 1414 to the outer surface 1416 and this angled portion can face outward relative to the surgical staple 1400.

The second tine 1406 can include a proximal end 1420 and a distal end 1422. Moreover, the second tine 1406 can include an inner surface 1424 and an outer surface 1426. In a particular embodiment, the inner surface 1424 of the second tine 1406 can be relatively longer than the outer surface 1426 of the second tine 1406. As such, the distal end 1422 of the second tine 1406 can be angled from the inner surface 1424 to the outer surface 1426 and this angled portion can face outward relative to the surgical staple 1400.

As illustrated in FIG. 15, the distal end 1412 of the first tine 1404 can include a necked portion 1430 that leads to an enlarged portion 1432. The enlarged portion 1432 can be generally rectangular. Alternatively, the enlarged portion 1432 can be generally square. FIG. 16 indicates that the distal end 1422 of the second tine 1406 can also include a necked portion 1440 that leads to an enlarged portion 1442. The enlarged portion 1442 can be generally rectangular. Alternatively, the enlarged portion 1442 can be generally square. In a particular embodiment, the enlarged portion 1432, 1442 of the distal end 1412, 1422 of each tine 1404, 1406 can provide greater surface area for engaging tissue, e.g., bone or flesh, after the surgical staple 1400 is installed in a patient. Further, the enlarged portion 1432, 1442 of the distal end 1412, 1422 of each tine 1404, 1406 can substantially reduce bone plowing.

The surgical staple 1400 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. In a particular embodiment, the surgical staple 1400 can be moved between a closed configuration and an open configuration or between an open configuration and a closed configuration, described above. Alternatively, the surgical staple 1400 can be moved between a straight configuration and a twisted configuration, described above.

### Description of a Sixth Embodiment of a Two-Tine Surgical Staple

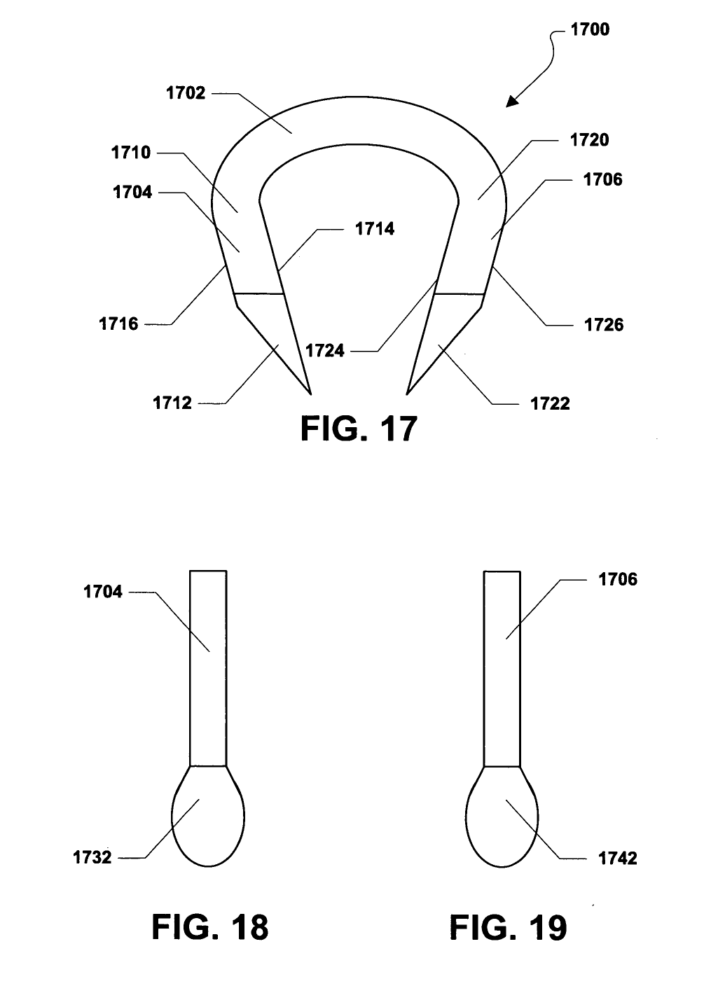

Referring to FIG. 17 through FIG. 19, a sixth embodiment of a two-tine surgical staple is shown and is generally designated 1700. As shown, the two-tine surgical staple 1700 can be generally U-shaped and can include a base 1702. A first tine 1704 and a second tine 1706 can extend from the base 1702.

As depicted, the first tine 1704 can include a proximal end 1710 and a distal end 1712. Moreover, the first tine 1704 can include an inner surface 1714 and an outer surface 1716. In a particular embodiment, the inner surface 1714 of the first tine 1704 can be relatively longer than the outer surface 1716 of the first tine 1704. As such, the distal end 1712 of the first tine 1704 can be angled from the inner surface 1714 to the outer surface 1716 and this angled portion can face outward relative to the surgical staple 1700.

The second tine 1706 can include a proximal end 1720 and a distal end 1722. Moreover, the second tine 1706 can include an inner surface 1724 and an outer surface 1726. In a particular embodiment, the inner surface 1724 of the second tine 1706 can be relatively longer than the outer surface 1726 of the second tine 1706. As such, the distal end 1722 of the second tine 1706 can be angled from the inner surface 1724 to the outer surface 1726 and this angled portion can face outward relative to the surgical staple 1700.

As illustrated in FIG. 18, the distal end 1712 of the first tine 1704 can include an enlarged portion 1732. The enlarged portion 1732 can be generally elliptical. Alternatively, the enlarged portion 1732 can be generally circular. FIG. 19 indicates that the distal end 1722 of the second tine 1706 can also include an enlarged portion 1742. The enlarged portion 1742 can be generally elliptical. Alternatively, the enlarged portion 1742 can be generally circular. In a particular embodiment, the enlarged portion 1732, 1742 of the distal end 1712, 1722 of each tine 1704, 1706 can provide greater surface area for engaging tissue, e.g., bone or flesh, after the surgical staple 1700 is installed in a patient. Further, the enlarged portion 1732, 1742 of the distal end 1712, 1722 of each tine 1704, 1706 can substantially reduce bone plowing.

The surgical staple 1700 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. In a particular embodiment, the surgical staple 1700 can be moved between a closed configuration and an open configuration or between an open configuration and a closed configuration, described above. Alternatively, the surgical staple 1700 can be moved between a straight configuration and a twisted configuration, described above.

### Description of a Seventh Embodiment of a Two-Tine Surgical Staple

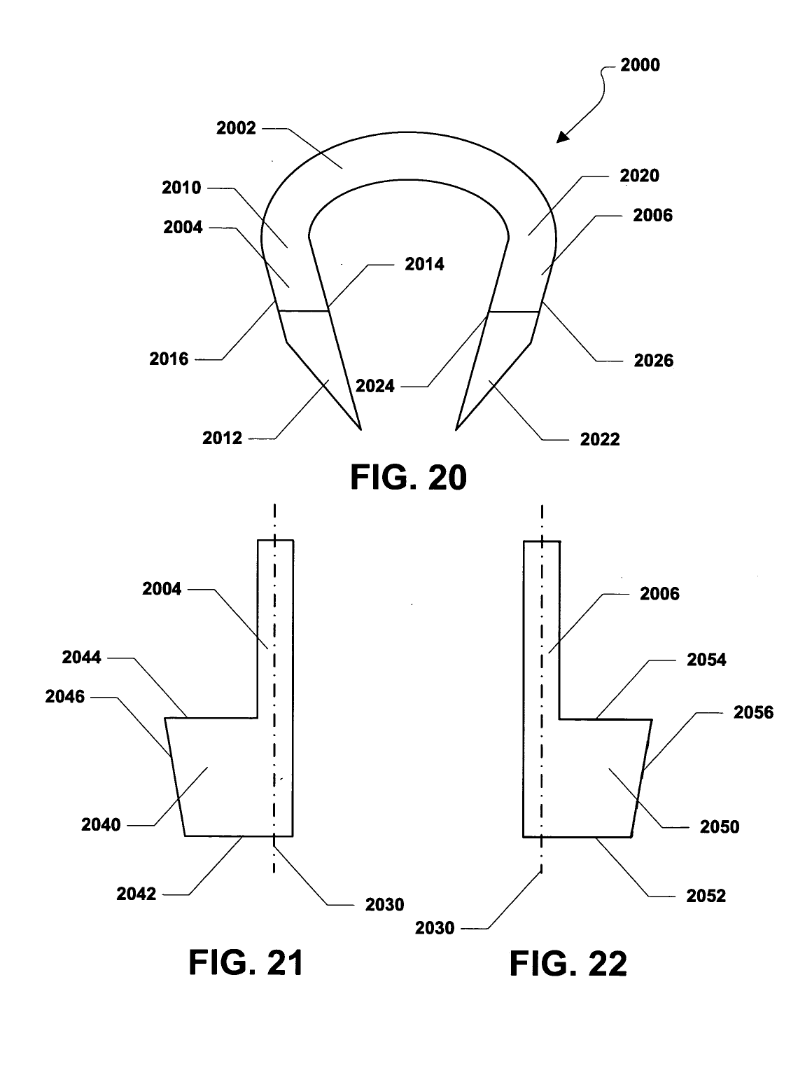

Referring to FIG. 20 through FIG. 22, a seventh embodiment of a two-tine surgical staple is shown and is generally designated 2000. As shown, the two-tine surgical staple 2000 can be generally U-shaped and can include a base 2002. A first tine 2004 and a second tine 2006 can extend from the base 2002.

As depicted, the first tine 2004 can include a proximal end 2010 and a distal end 2012. Moreover, the first tine 2004 can include an inner surface 2014 and an outer surface 2016. In a particular embodiment, the inner surface 2014 of the first tine 2004 can be relatively longer than the outer surface 2016 of the first tine 2004. As such, the distal end 2012 of the first tine 2004 can be angled from the inner surface 2014 to the outer surface 2016 and this angled portion can face outward relative to the surgical staple 2000.

The second tine 2006 can include a proximal end 2020 and a distal end 2022. Moreover, the second tine 2006 can include an inner surface 2024 and an outer surface 2026. In a particular embodiment, the inner surface 2024 of the second tine 2006 can be relatively longer than the outer surface 2026 of the second tine 2006. As such, the distal end 2022 of the second tine 2006 can be angled from the inner surface 2024 to the outer surface 2026 and this angled portion can face outward relative to the surgical staple 2000. In a particular embodiment, the surgical staple 2000 can include a longitudinal axis 2030.

As illustrated in FIG. 21, the distal end 2012 of the first tine 2004 can include an enlarged portion 2040. The enlarged portion 2040 can include a first surface 2042 that can be substantially perpendicular to the longitudinal axis 2030. Also, the enlarged portion 2040 can include a second surface 2044 that can be substantially perpendicular to the longitudinal axis 2030. In a particular embodiment, the first surface 2042 can be spaced from the second surface 2044 and a third surface 2046 can extend therebetween. In a particular embodiment, the third surface 2046 can be angled with respect to the longitudinal axis 2030.

As illustrated in FIG. 22, the distal end 2022 of the second tine 2006 can include an enlarged portion 2050. The enlarged portion 2050 can include a first surface 2052 that can be substantially perpendicular to the longitudinal axis 2030. Also, the enlarged portion 2050 can include a second surface 2054 that can be substantially perpendicular to the longitudinal axis 2030. In a particular embodiment, the first surface 2052 can be spaced from the second surface 2054 and a third surface 2056 can extend therebetween. In a particular embodiment, the third surface 2056 can be angled with respect to the longitudinal axis 2030.

In a particular embodiment, the enlarged portion 2040, 2050 of the distal end 2012, 2022 of each tine 2004, 2006 can provide greater surface area for engaging tissue, e.g., bone or flesh, after the surgical staple 2000 is installed in a patient. Further, the enlarged portion 2032, 2042 of the distal end 2012, 2022 of each tine 2004, 2006 can substantially reduce bone plowing.

The surgical staple 2000 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. In a particular embodiment, the surgical staple 2000 can be moved between a closed configuration and an open configuration or between an open configuration and a closed configuration, described above. Alternatively, the surgical staple 2000 can be moved between a straight configuration and a twisted configuration, described above.

### Description of an Eighth Embodiment of a Two-Tine Surgical Staple

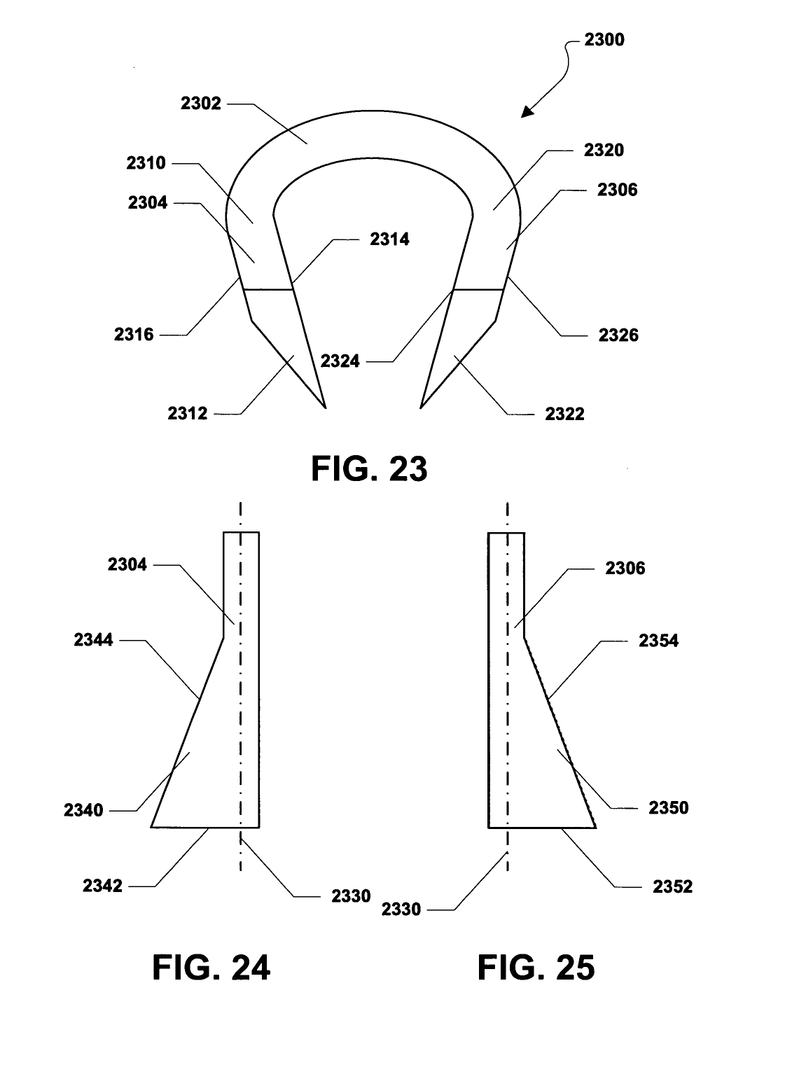

Referring to FIG. 23 through FIG. 25, an eighth embodiment of a two-tine surgical staple is shown and is generally designated 2300. As shown, the two-tine surgical staple 2300 can be generally U-shaped and can include a base 2302. A first tine 2304 and a second tine 2306 can extend from the base 2302.

As depicted, the first tine 2304 can include a proximal end 2310 and a distal end 2312. Moreover, the first tine 2304 can include an inner surface 2314 and an outer surface 2316. In a particular embodiment, the inner surface 2314 of the first tine 2304 can be relatively longer than the outer surface 2316 of the first tine 2304. As such, the distal end 2312 of the first tine 2304 can be angled from the inner surface 2314 to the outer surface 2316 and this angled portion can face outward relative to the surgical staple 2300.

The second tine 2306 can include a proximal end 2320 and a distal end 2322. Moreover, the second tine 2306 can include an inner surface 2324 and an outer surface 2326. In a particular embodiment, the inner surface 2324 of the second tine 2306 can be relatively longer than the outer surface 2326 of the second tine 2306. As such, the distal end 2322 of the second tine 2306 can be angled from the inner surface 2324 to the outer surface 2326 and this angled portion can face outward relative to the surgical staple 2300. In a particular embodiment, the surgical staple 2300 can include a longitudinal axis 2330.

As illustrated in FIG. 24, the distal end 23 12 of the first tine 2304 can include an enlarged portion 2340. The enlarged portion 2340 can include a first surface 2342 that can be substantially perpendicular to the longitudinal axis 2330. Also, the enlarged portion 2340 can include a second surface 2344 that can extend between the first surface 2342 and the proximal end 2310 of the first tine 2304. As shown, the second surface 2344 can be angled with respect to the longitudinal axis 2330.

As illustrated in FIG. 25, the distal end 2322 of the second tine 2306 can include an enlarged portion 2350. The enlarged portion 2350 can include a first surface 2352 that can be substantially perpendicular to the longitudinal axis 2330. Also, the enlarged portion 2350 can include a second surface 2354 that can extend between the first surface 2352 and the proximal end 2320 of the second tine 2306. As shown, the second surface 2354 can be angled with respect to the longitudinal axis 2330.

In a particular embodiment, the enlarged portion 2340, 2350 of the distal end 2312, 2322 of each tine 2304, 2306 can provide greater surface area for engaging tissue, e.g., bone or flesh, after the surgical staple 2300 is installed in a patient. Further, the enlarged portion 2332, 2342 of the distal end 2312, 2322 of each tine 2304, 2306 can substantially reduce bone plowing.

The surgical staple 2300 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. In a particular embodiment, the surgical staple 2300 can be moved between a closed configuration and an open configuration or between an open configuration and a closed configuration, described above. Alternatively, the surgical staple 2300 can be moved between a straight configuration and a twisted configuration, described above.

### Description of a Ninth Embodiment of a Two-Tine Surgical Staple

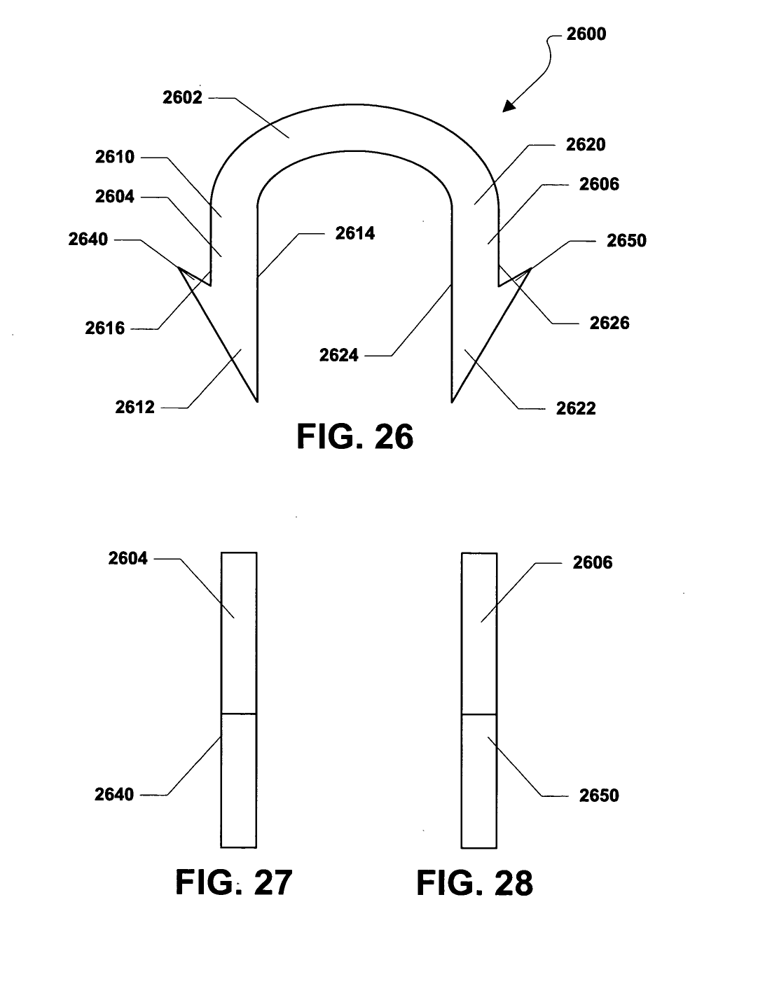

Referring to FIG. 26 through FIG. 28, an eighth embodiment of a two-tine surgical staple is shown and is generally designated 2600. As shown, the two-tine surgical staple 2600 can be generally U-shaped and can include a base 2602. A first tine 2604 and a second tine 2606 can extend from the base 2602.

As depicted, the first tine 2604 can include a proximal end 2610 and a distal end 2612. Moreover, the first tine 2604 can include an inner surface 2614 and an outer surface 2616. In a particular embodiment, the inner surface 2614 of the first tine 2604 can be relatively longer than the outer surface 2616 of the first tine 2604. As such, the distal end 2612 of the first tine 2604 can be angled from the inner surface 2614 to the outer surface 2616 and this angled portion can face outward relative to the surgical staple 2600.

The second tine 2606 can include a proximal end 2620 and a distal end 2622. Moreover, the second tine 2606 can include an inner surface 2624 and an outer surface 2626. In a particular embodiment, the inner surface 2624 of the second tine 2606 can be relatively longer than the outer surface 2626 of the second tine 2606. As such, the distal end 2622 of the second tine 2606 can be angled from the inner surface 2624 to the outer surface 2626 and this angled portion can face outward relative to the surgical staple 2600. In a particular embodiment, the surgical staple 2600 can include a longitudinal axis 2630.

As illustrated in FIG. 27, the distal end 2612 of the first tine 2604 can include a barb 2640. The barb 2640 can extend from the distal end 2612 of the first tine 2604 at an angle with respect to the longitudinal axis 2630. Further, the barb 2640 of the first tine 2604 can extend outward relative to the surgical staple 2600.

As illustrated in FIG. 28, the distal end 2622 of the second tine 2606 can include a barb 2650. The barb 2650 can extend from the distal end 2622 of the second tine 2606 at an angle with respect to the longitudinal axis 2630. Further, the barb 2650 of the second tine 2606 can extend outward relative to the surgical staple 2600.

In a particular embodiment, the barb 2640, 2650 that extends from the distal end 2612, 2622 of each tine 2604, 2606 can engage tissue, e.g., bone or flesh, after the surgical staple 2600 is installed in a patient and substantially prevent the surgical staple 2600 from prematurely withdrawing from the tissue.

The surgical staple 2600 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. In a particular embodiment, the surgical staple 2600 can be moved between a closed configuration and an open configuration or between an open configuration and a closed configuration, described above. Alternatively, the surgical staple 2600 can be moved between a straight configuration and a twisted configuration, described above.

### Description of a Tenth Embodiment of a Two-Tine Surgical Staple

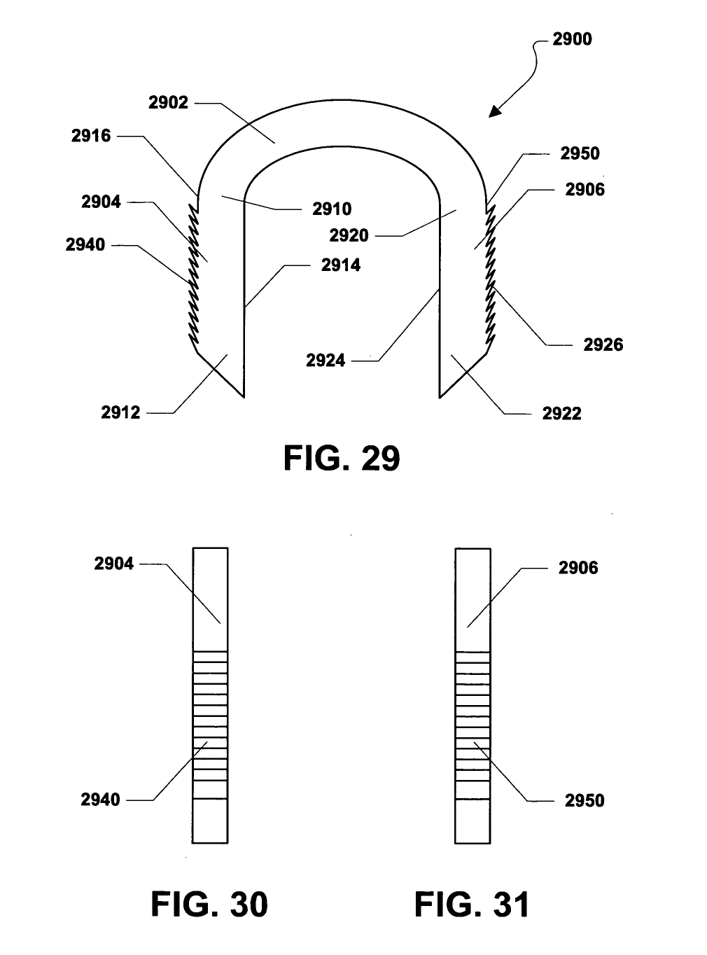

Referring to FIG. 29 through FIG. 31, a tenth embodiment of a two-tine surgical staple is shown and is generally designated 2900. As shown, the two-tine surgical staple 2900 can be generally U-shaped and can include a base 2902. A first tine 2904 and a second tine 2906 can extend from the base 2902.

As depicted, the first tine 2904 can include a proximal end 2910 and a distal end 2912. Moreover, the first tine 2904 can include an inner surface 2914 and an outer surface 2916. In a particular embodiment, the inner surface 2914 of the first tine 2904 can be relatively longer than the outer surface 2916 of the first tine 2904. As such, the distal end 2912 of the first tine 2904 can be angled from the inner surface 2914 to the outer surface 2916 and this angled portion can face outward relative to the surgical staple 2900.

The second tine 2906 can include a proximal end 2920 and a distal end 2922. Moreover, the second tine 2906 can include an inner surface 2924 and an outer surface 2926. In a particular embodiment, the inner surface 2924 of the second tine 2906 can be relatively longer than the outer surface 2926 of the second tine 2906. As such, the distal end 2922 of the second tine 2906 can be angled from the inner surface 2924 to the outer surface 2926 and this angled portion can face outward relative to the surgical staple 2900. In a particular embodiment, the surgical staple 2900 can include a longitudinal axis 2930.

As illustrated in FIG. 30, the first tine 2904 can include a plurality of barbs 2940 that can extend substantially along the length of the first tine 2904. Each of the barbs 2940 can extend from the first tine 2904 at an angle with respect to the longitudinal axis 2930. Further, each of the barbs 2940 of the first tine 2904 can extend outward relative to the surgical staple 2900.

As illustrated in FIG. 31, the second tine 2906 can include a plurality of barbs 2950 that can extend substantially along the length of the second tine 2906. Each of the barbs 2950 can extend from the second tine 2906 at an angle with respect to the longitudinal axis 2930. Further, each of the barbs 2950 of the second tine 2906 can extend outward relative to the surgical staple 2900.

In a particular embodiment, the barbs 2940, 2950 that extend from each tine 2904, 2906 can engage tissue, e.g., bone or flesh, after the surgical staple 2900 is installed in a patient and substantially prevent the surgical staple 2900 from prematurely withdrawing from the tissue.

The surgical staple 2900 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. In a particular embodiment, the surgical staple 2900 can be moved between a closed configuration and an open configuration or between an open configuration and a closed configuration, described above. Alternatively, the surgical staple 2900 can be moved between a straight configuration and a twisted configuration, described above.

### Description of an Eleventh Embodiment of a Two-Tine Surgical Staple

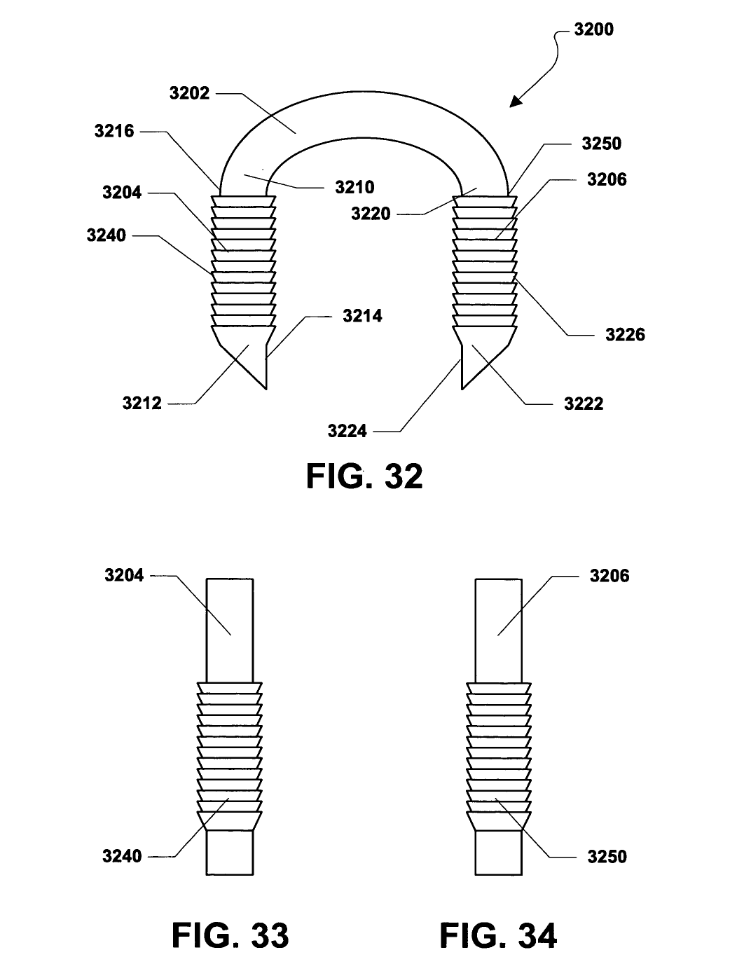

Referring to FIG. 32 through FIG. 34, a tenth embodiment of a two-tine surgical staple is shown and is generally designated 3200. As shown, the two-tine surgical staple 3200 can be generally U-shaped and can include a base 3202. A first tine 3204 and a second tine 3206 can extend from the base 3202.

As depicted, the first tine 3204 can include a proximal end 3210 and a distal end 3212. Moreover, the first tine 3204 can include an inner surface 3214 and an outer surface 3216. In a particular embodiment, the inner surface 3214 of the first tine 3204 can be relatively longer than the outer surface 3216 of the first tine 3204. As such, the distal end 3212 of the first tine 3204 can be angled from the inner surface 3214 to the outer surface 3216 and this angled portion can face outward relative to the surgical staple 3200.

The second tine 3206 can include a proximal end 3220 and a distal end 3222. Moreover, the second tine 3206 can include an inner surface 3224 and an outer surface 3226. In a particular embodiment, the inner surface 3224 of the second tine 3206 can be relatively longer than the outer surface 3226 of the second tine 3206. As such, the distal end 3222 of the second tine 3206 can be angled from the inner surface 3224 to the outer surface 3226 and this angled portion can face outward relative to the surgical staple 3200. In a particular embodiment, the surgical staple 3200 can include a longitudinal axis 3230.

As illustrated in FIG. 33, the first tine 3204 can include a plurality of rings 3240. Each ring 3240 can circumscribe the first tine 3204. Further, each of the rings 3240 of the first tine 3204 can extend outward relative to the surgical staple 3200. Moreover, the plurality of rings 3240 can extend substantially along the length of the first tine 3204.

As illustrated in FIG. 34, the second tine 3206 can include a plurality of rings 3250. Each ring 3250 can circumscribe the second tine 3206. Further, each of the rings 3250 of the second tine 3206 can extend outward relative to the second tine 3206. Moreover, the plurality of rings 3250 can extend substantially along the length of the second tine 3206.

In a particular embodiment, the rings 3240, 3250 that extend from each tine 3204, 3206 can engage tissue, e.g., bone or flesh, after the surgical staple 3200 is installed in a patient and substantially prevent the surgical staple 3200 from prematurely withdrawing from the tissue.

The surgical staple 3200 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. In a particular embodiment, the surgical staple 3200 can be moved between a closed configuration and an open configuration or between an open configuration and a closed configuration, described above. Alternatively, the surgical staple 3200 can be moved between a straight configuration and a twisted configuration, described above.

### Description of a Twelfth Embodiment of a Two-Tine Surgical Staple

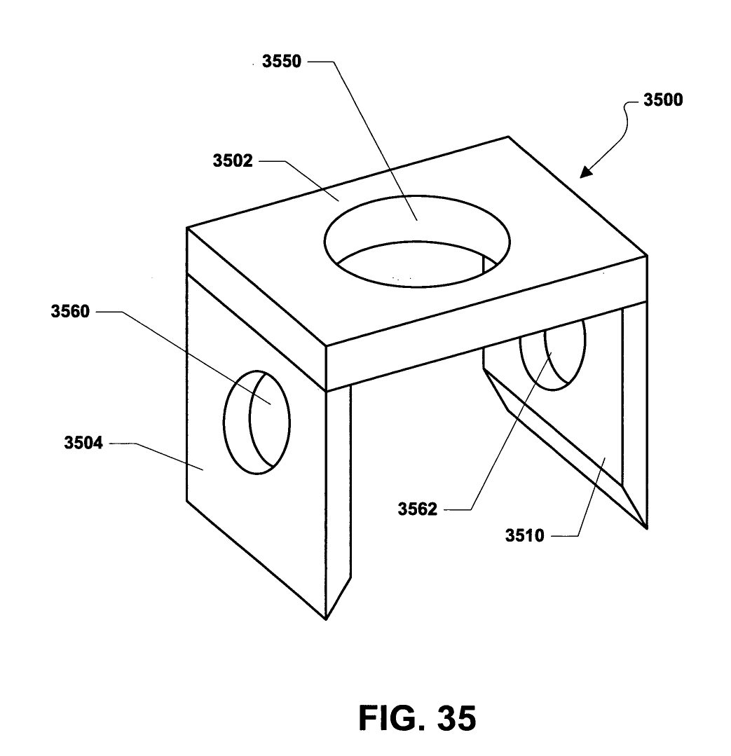

Referring initially to FIG. 35 through FIG. 40, a twelfth embodiment of a two-tine surgical staple is shown and is generally designated 3500. As shown, the two-tine surgical staple 3500 can include a base 3502. A first tine 3504 and a second tine 3506 can extend from the base 3502. For example, the first tine 3504 can extend from one end of the base 3502 and the second tine 3506 can extend from the other end of the base 3502. In a particular embodiment, the tines 3504, 3506 can extend substantially perpendicular from the base 3502.

As depicted, the first tine 3504 can include a proximal end 3520 and a distal end 3522. Moreover, the first tine 3504 can include an inner surface 3524 and an outer surface 3526. In a particular embodiment, the inner surface 3524 of the first tine 3504 can be relatively shorter than the outer surface 3526 of the first tine 3504. As such, the distal end 3522 of the first tine 3504 can be angled from the inner surface 3524 to the outer surface 3526 and this angled portion can face inward relative to the surgical staple 3500.

The second tine 3506 can include a proximal end 3530 and a distal end 3532. Moreover, the second tine 3506 can include an inner surface 3534 and an outer surface 3536. In a particular embodiment, the inner surface 3534 of the second tine 3506 can be relatively shorter than the outer surface 3536 of the second tine 3506. As such, the distal end 3532 of the second tine 3506 can be angled from the inner surface 3534 to the outer surface 3536 and this angled portion can face inward relative to the surgical staple 3500.

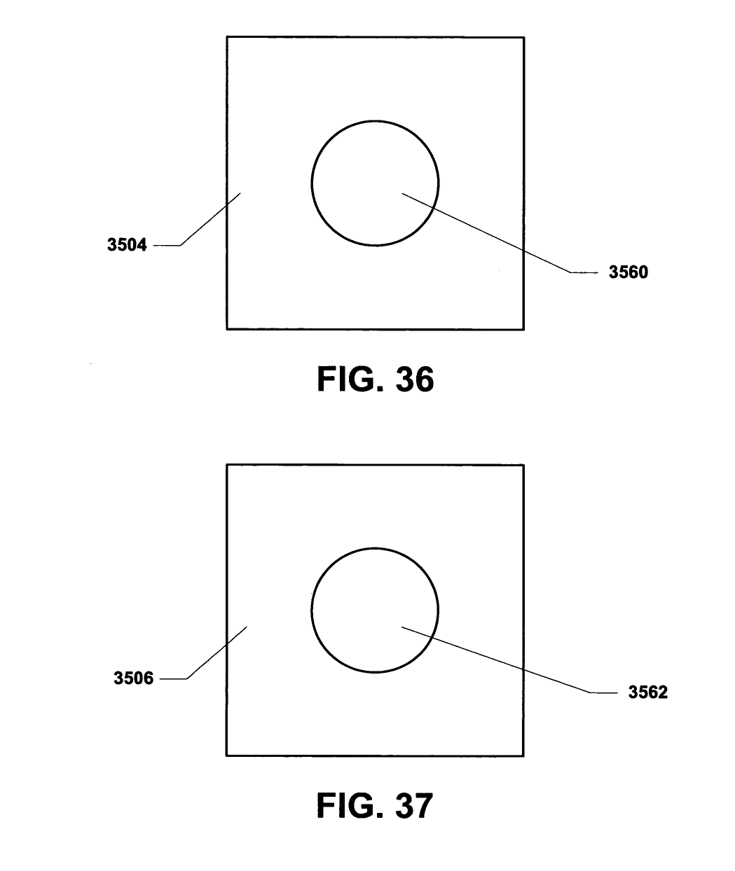

FIG. 35 indicates that the base 3502 of the surgical staple 3500 can be generally rectangular. Further, the base 3502 of the surgical staple 3500 can be formed with a hole 3550. In a particular embodiment, the hole 3550 can facilitate retrieval and insertion of the surgical staple 3500. Further, as shown in FIG. 36 and FIG. 37, the first tine 3504 can be formed with a hole 3560 and the second tine 3506 can be formed with a hole 3562. The holes 3560, 3562 in the tines 3504, 3506 can also facilitate retrieval and insertion of the surgical staple 3500. Further, the holes 3560, 3562 can engage tissue when the surgical staple 3500 is installed in a patient and the surgical staple 3500 is moved to an open configuration, described below.

The surgical staple 3500 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol.

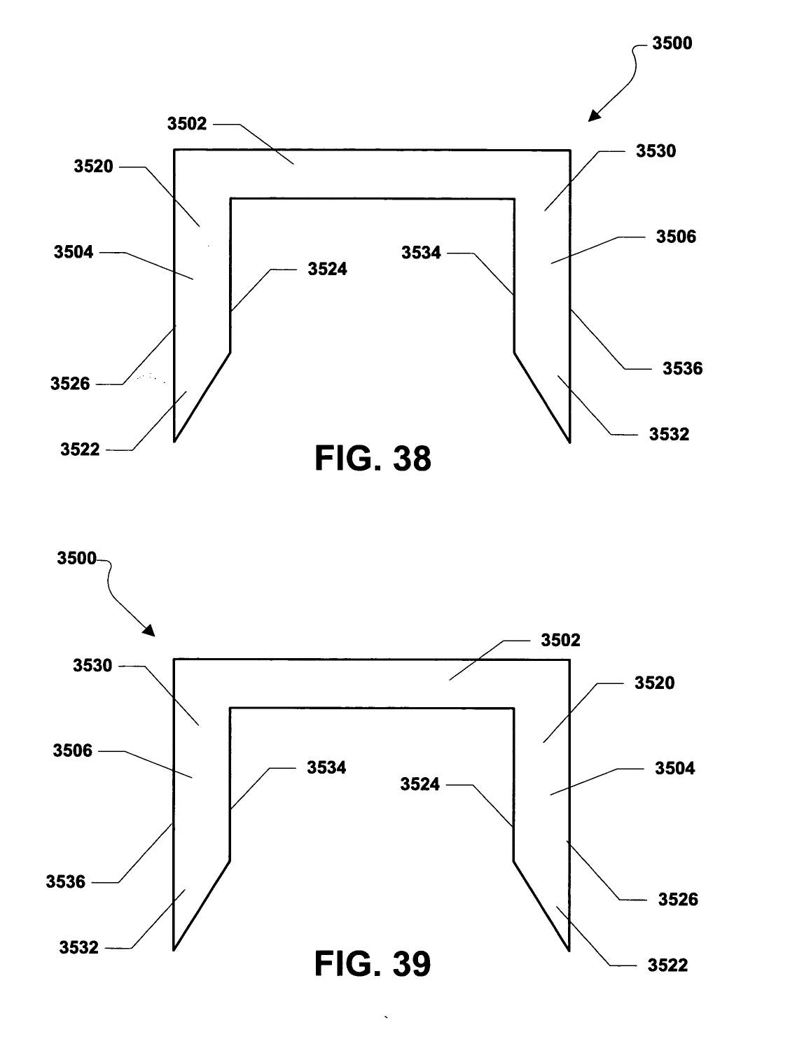

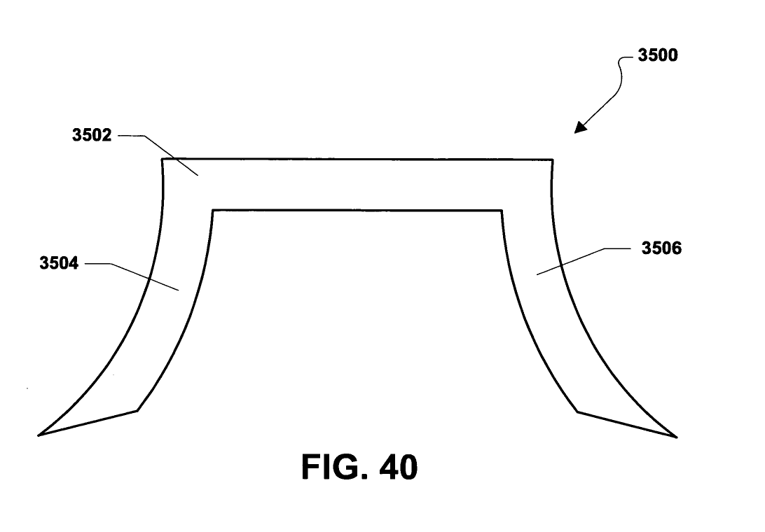

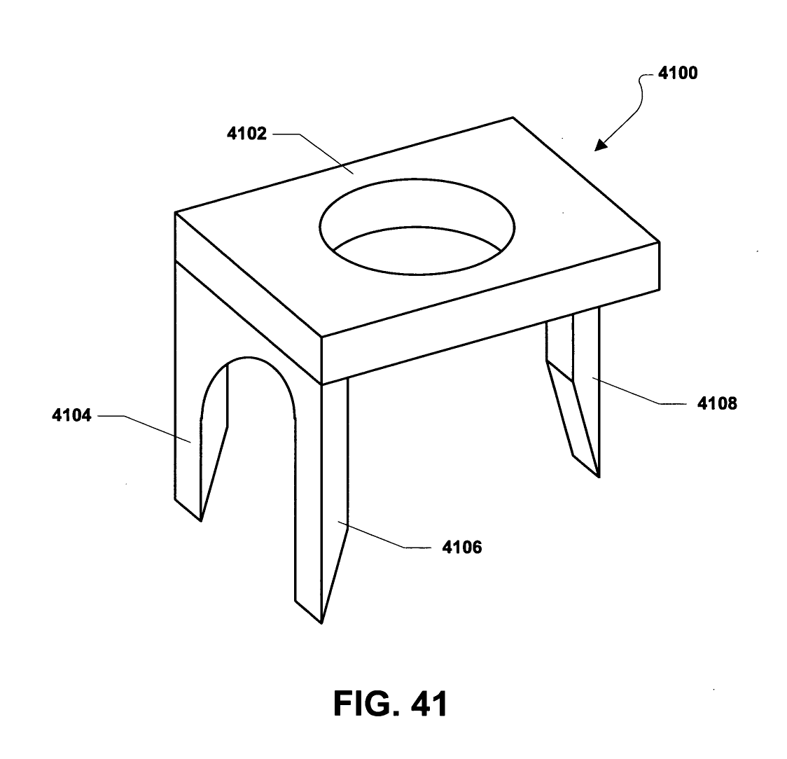

The surgical staple 3500 can be moved between a closed configuration, shown in FIG. 38 and FIG. 39, and an open configuration, shown in FIG. 40. In the closed configuration, the tines 3504, 3506 of the surgical staple 3500 can be substantially straight and substantially perpendicular to the base 3502 of the surgical staple 3500.

In the open configuration, the tines 3504, 3506 of the surgical staple 3500 can be bent outward relative to the base 3502 of the surgical staple 3500. Further, the tines 3504, 3506 can bend outward relative to each other.

In a particular embodiment, the surgical staple 3500 can be moved from the open configuration to the closed configuration by applying heat to the surgical staple 3500.

### Description of a First Embodiment of a Three-Tine Surgical Staple

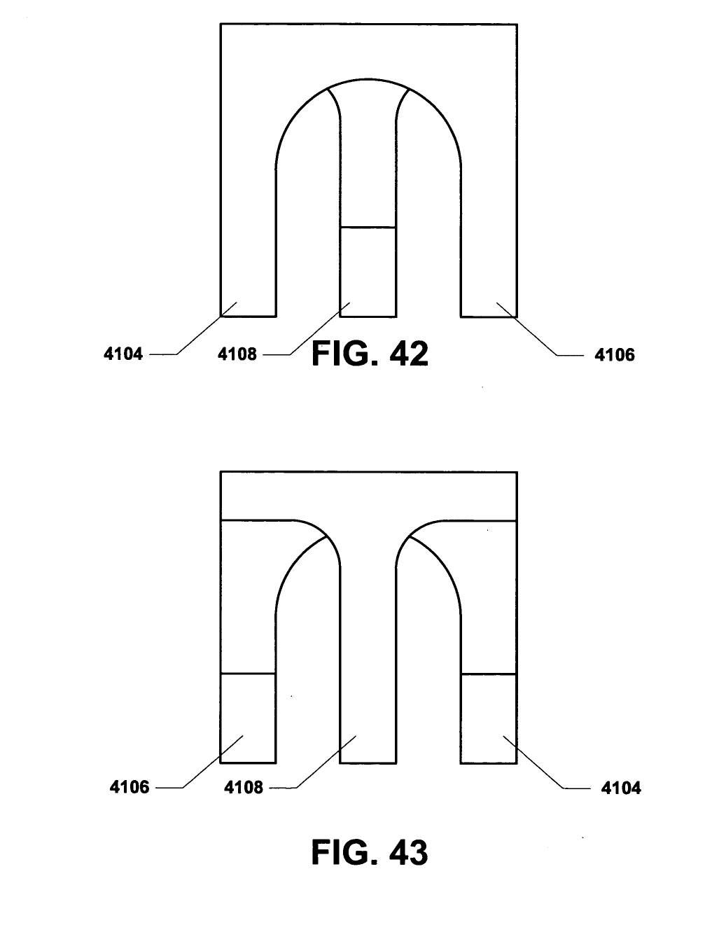

Referring initially to FIG. 41 through FIG. 47, a first embodiment of a three-tine surgical staple is shown and is generally designated 4100. As shown, the three-tine surgical staple 4100 can include a base 4102. A first tine 4104, a second tine 4106, and a third tine 4108 can extend from the base 4102. In a particular embodiment, the tines 4104, 4106, 4108 can extend substantially perpendicular from the base 4102.

As depicted, the first tine 4104 can include a proximal end 4110 and a distal end 4112. Moreover, the first tine 4104 can include an inner surface 4114 and an outer surface 4116. In a particular embodiment, the inner surface 4114 of the first tine 4104 can be relatively shorter than the outer surface 4116 of the first tine 4104. As such, the distal end 4112 of the first tine 4104 can be angled from the inner surface 4114 to the outer surface 4116 and this angled portion can face inward relative to the surgical staple 4100.

The second tine 4106 can include a proximal end 4120 and a distal end 4122. Moreover, the second tine 4106 can include an inner surface 4124 and an outer surface 4126. In a particular embodiment, the inner surface 4124 of the second tine 4106 can be relatively shorter than the outer surface 4126 of the second tine 4106. As such, the distal end 4122 of the second tine 4106 can be angled from the inner surface 4124 to the outer surface 4126 and this angled portion can face inward relative to the surgical staple 4100.

The third tine 4108 can include a proximal end 4130 and a distal end 4132. Moreover, the third tine 4108 can include an inner surface 4134 and an outer surface 4136. In a particular embodiment, the inner surface 4134 of the third tine 4108 can be relatively shorter than the outer surface 4136 of the third tine 4108. As such, the distal end 4132 of the third tine 4108 can be angled from the inner surface 4134 to the outer surface 4136 and this angled portion can face inward relative to the surgical staple 4100.

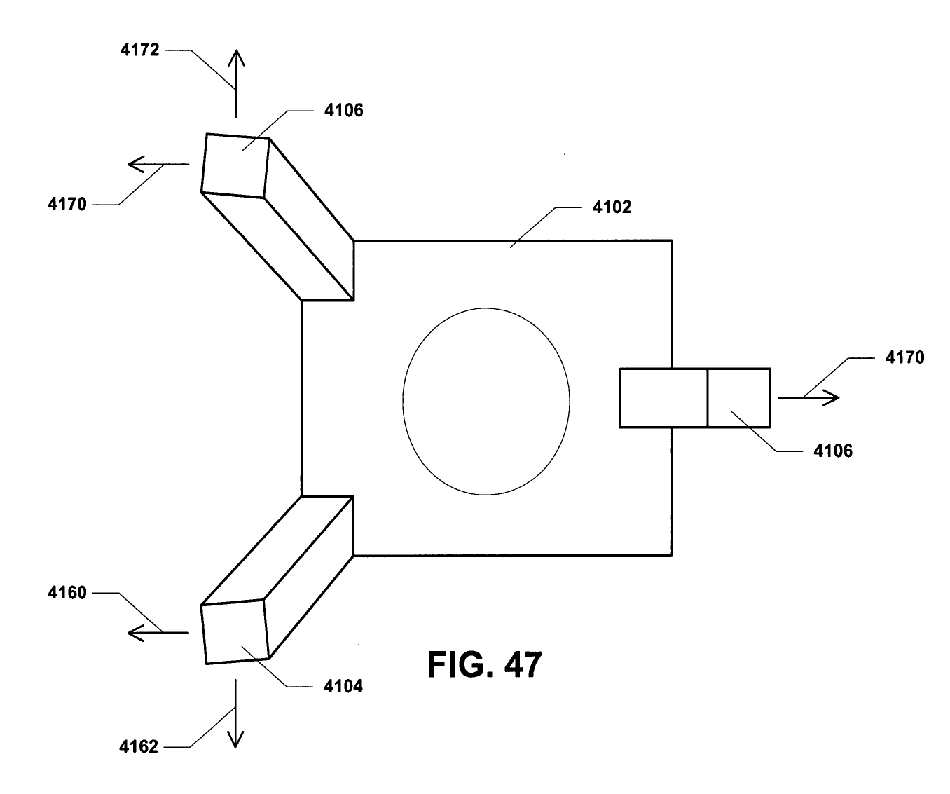

FIG. 41 and FIG. 47 indicate that the base 4102 of the surgical staple 4100 can be generally rectangular. Further, the base 4102 of the surgical staple 4100 can be formed with a hole 4140. In a particular embodiment, the hole 4140 can facilitate retrieval and insertion of the surgical staple 4100.

The surgical staple 4100 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol.

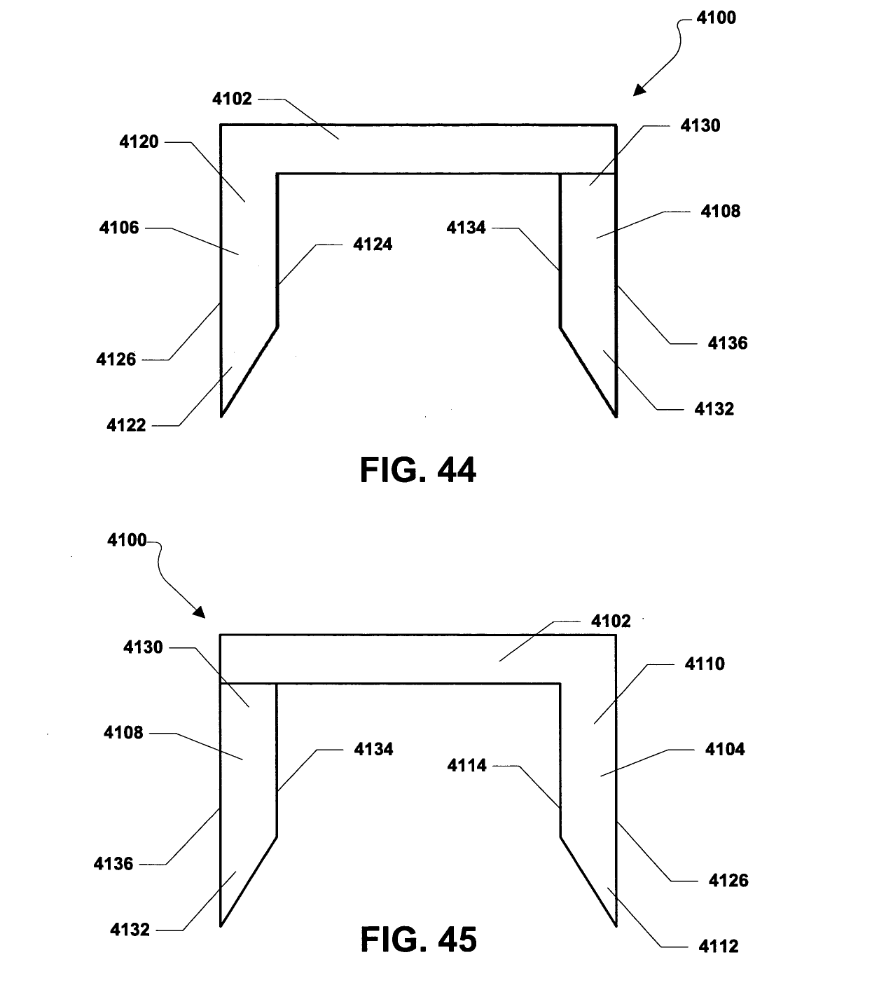

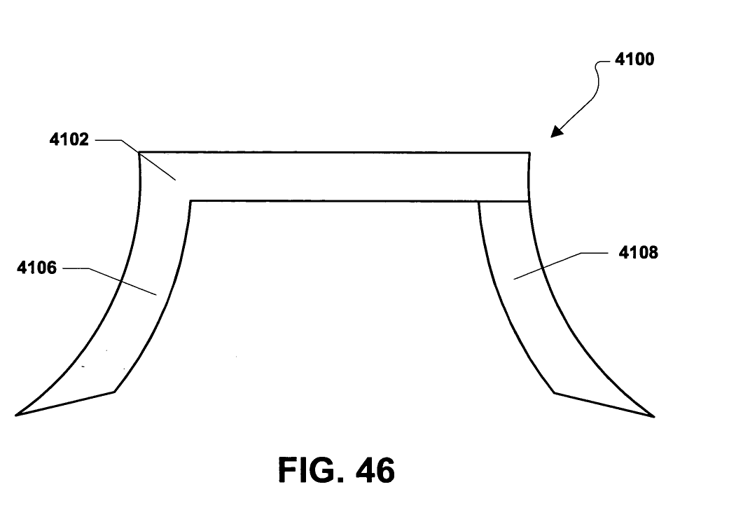

The surgical staple 4100 can be moved between a closed configuration, shown in FIG. 44 and FIG. 45, and an open configuration, shown in FIG. 46 and FIG. 47. In the closed configuration, the tines 4104, 4106, 4108 of the surgical staple 4100 can be substantially straight and substantially perpendicular to the base 4102 of the surgical staple 4100.

In the open configuration, the tines 4104, 4106, 4108 of the surgical staple 4100 can be bent outward relative to the base 4102 of the surgical staple 4100. In particular, the first tine 4104 can bend longitudinally with respect to the base 4102, as indicated by arrow 4160, and laterally with respect to the base 4102, as indicated by arrow 4162. Further, the second tine 4106 can bend longitudinally with respect to the base 4102, as indicated by arrow 4170, and laterally with respect to the base 4102, as indicated by arrow 4172. The third tine 4108 can move longitudinally with respect to the base 4102 as indicated by arrow 4180. Accordingly, the first tine 4104 and the second tine 4106 can bend outward relative to the third tine 4108 and the first tine 4104 and the second tine 4106 can be bend outward relative to each other.

In a particular embodiment, the surgical staple 4100 can be moved from the open configuration to the closed configuration by applying heat to the surgical staple 4100.

Further, in a particular embodiment, the first tine 4104, the second tine 4106, the third tine 4108, or a combination thereof can include one or more of the enlarged portions described herein.

### Description of a Second Embodiment of a Three-Tine Surgical Staple

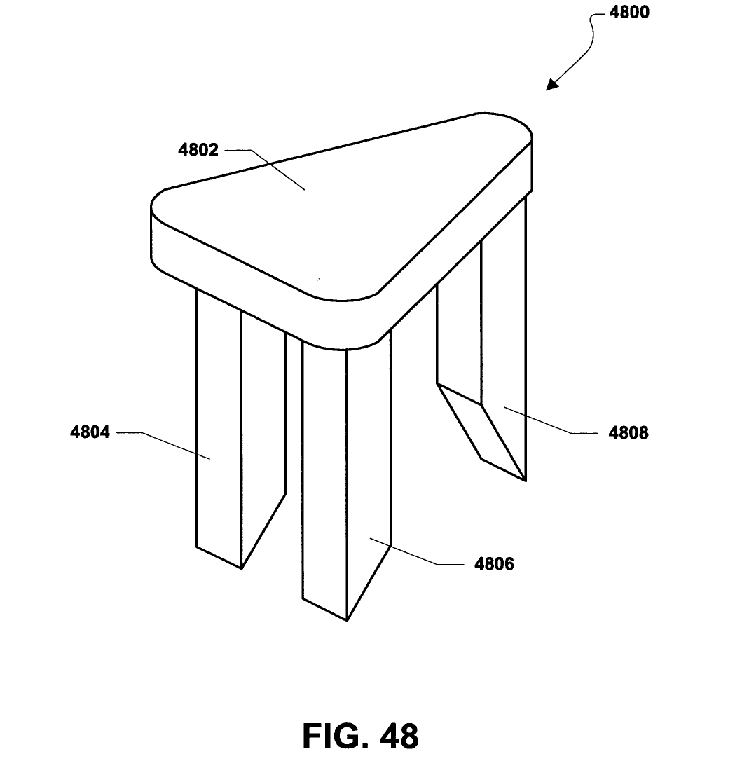

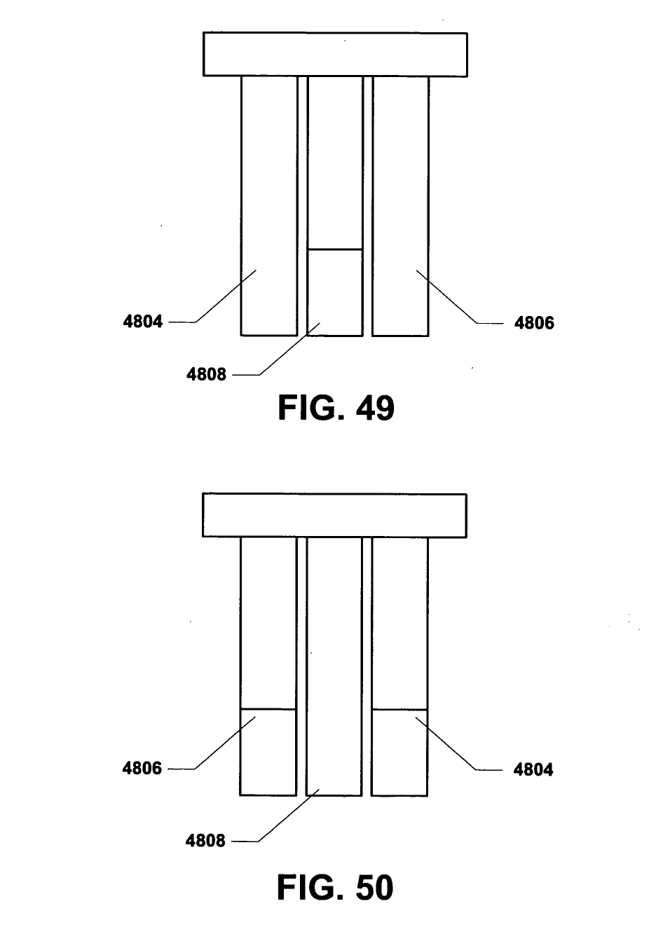

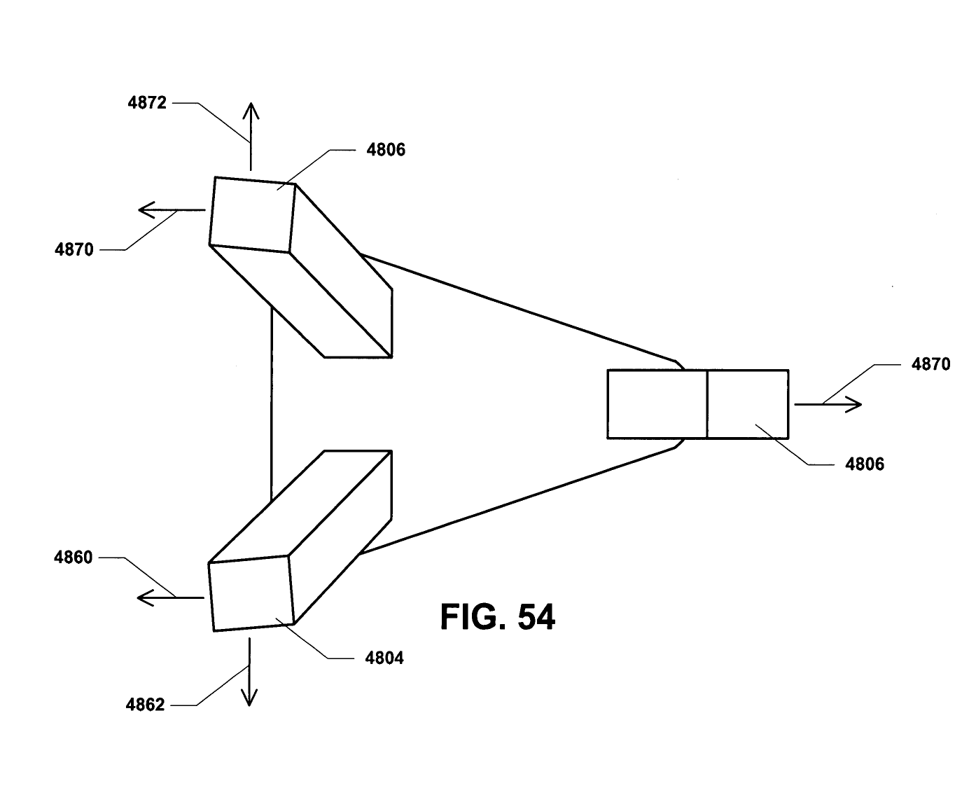

Referring initially to FIG. 48 through FIG. 54, a second embodiment of a three-tine surgical staple is shown and is generally designated 4800. As shown, the three-tine surgical staple 4800 can include a base 4802. In a particular embodiment, as illustrated in FIG. 48 and FIG. 54, the base 4802 of the surgical staple 4800 can be generally triangular. A first tine 4804, a second tine 4806, and a third tine 4808 can extend from the base 4802. For example, the first tine 4804 and the second tine 4806 can extend from one end of the base 4802 and the third tine 4808 can extend from the other end of the base 4802. In a particular embodiment, the tines 4804, 4806, 4808 can extend substantially perpendicular from the base 4802.

As depicted, the first tine 4804 can include a proximal end 4810 and a distal end 4812. Moreover, the first tine 4804 can include an inner surface 4814 and an outer surface 4816. In a particular embodiment, the inner surface 4814 of the first tine 4804 can be relatively shorter than the outer surface 4816 of the first tine 4804. As such, the distal end 4812 of the first tine 4804 can be angled from the inner surface 4814 to the outer surface 4816 and this angled portion can face inward relative to the surgical staple 4800.

The second tine 4806 can include a proximal end 4820 and a distal end 4822. Moreover, the second tine 4806 can include an inner surface 4824 and an outer surface 4826. In a particular embodiment, the inner surface 4824 of the second tine 4806 can be relatively shorter than the outer surface 4826 of the second tine 4806. As such, the distal end 4822 of the second tine 4806 can be angled from the inner surface 4824 to the outer surface 4826 and this angled portion can face inward relative to the surgical staple 4800.

The third tine 4808 can include a proximal end 4830 and a distal end 4832. Moreover, the third tine 4808 can include an inner surface 4834 and an outer surface 4836. In a particular embodiment, the inner surface 4834 of the third tine 4808 can be relatively shorter than the outer surface 4836 of the third tine 4808. As such, the distal end 4832 of the third tine 4808 can be angled from the inner surface 4834 to the outer surface 4836 and this angled portion can face inward relative to the surgical staple 4800.

The surgical staple 4800 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol.

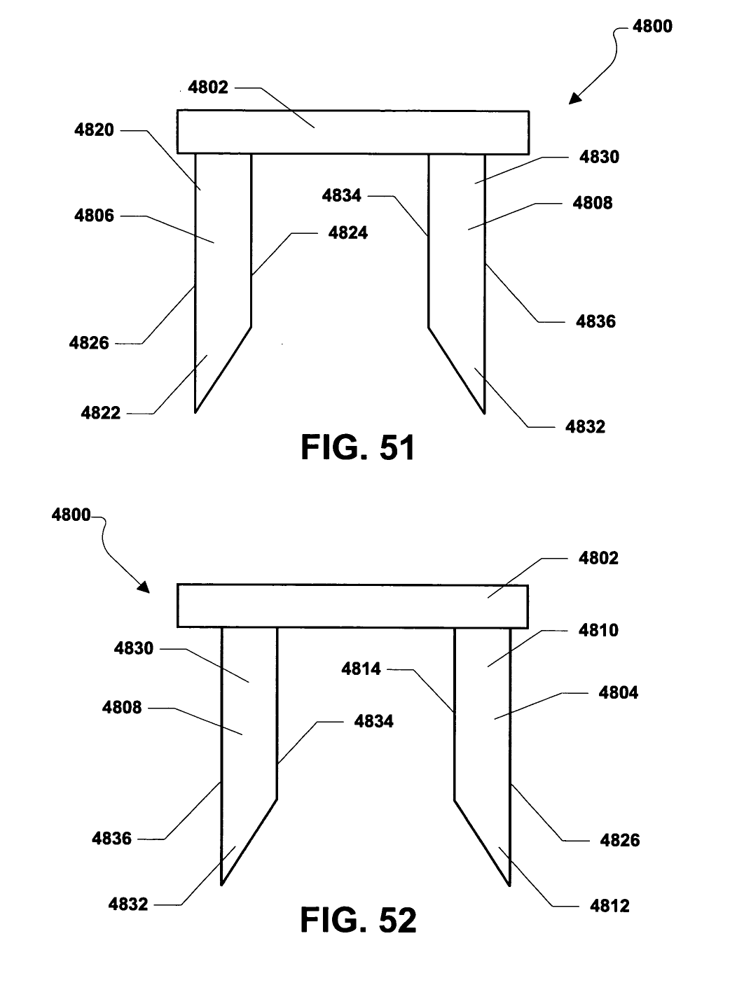

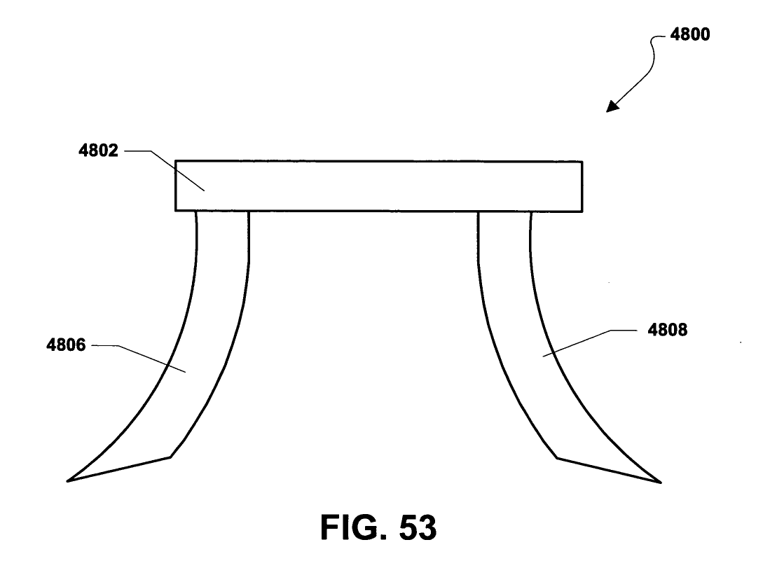

The surgical staple 4800 can be moved between a closed configuration, shown in FIG. 51 and FIG. 52, and an open configuration, shown in FIG. 53 and FIG. 54. In the closed configuration, the tines 4804, 4806, 4808 of the surgical staple 4800 can be substantially straight and substantially perpendicular to the base 4802 of the surgical staple 4800.

In the open configuration, the tines 4804, 4806, 4808 of the surgical staple 4800 can be bent outward relative to the base 4802 of the surgical staple 4800. In particular, the first tine 4804 can bend longitudinally with respect to the base 4802, as indicated by arrow 4860, and laterally with respect to the base 4802, as indicated by arrow 4862. Further, the second tine 4806 can bend longitudinally with respect to the base 4802, as indicated by arrow 4870, and laterally with respect to the base 4802, as indicated by arrow 4872. Accordingly, the first tine 4804 and the second tine 4806 can bend outward relative to the third tine 4808 and the first tine 4804 and the second tine 4806 can be bend outward relative to each other.

In a particular embodiment, the surgical staple 4800 can be moved from the open configuration to the closed configuration by applying heat to the surgical staple 4800.

Further, in a particular embodiment, the first tine 4804, the second tine 4806, the third tine 4808, or a combination thereof can include one or more of the enlarged portions described herein.

### Description of a First Embodiment of a Four-Tine Surgical Staple

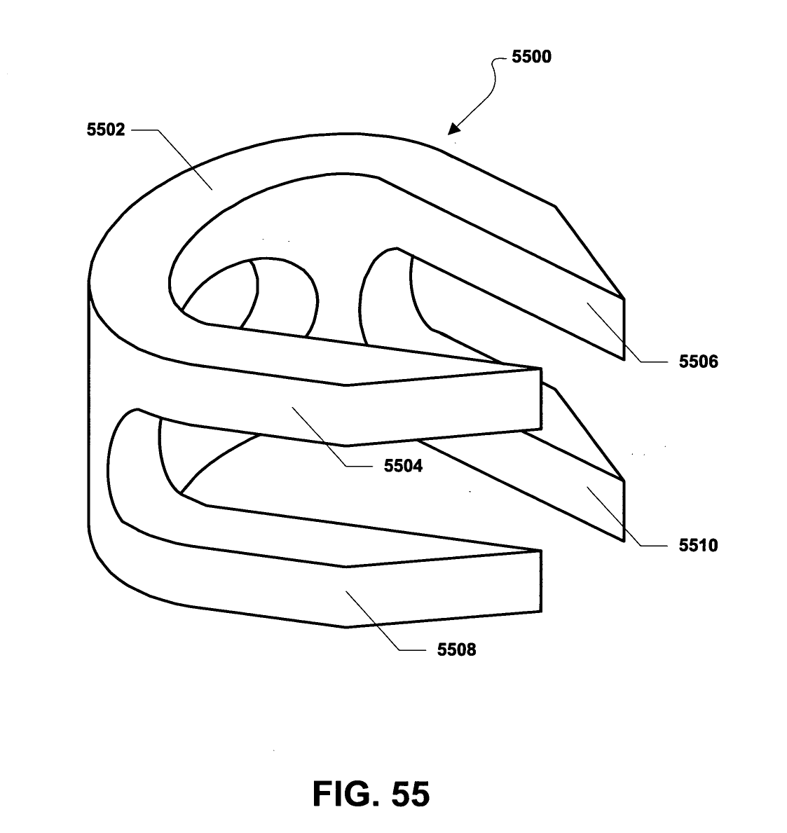

Referring initially to FIG. 55 through FIG. 58, a first embodiment of a four-tine surgical staple is shown and is generally designated 5500. As shown, the four-tine surgical staple 5500 can be generally U-shaped and can include a base 5502. A first tine 5504, a second tine 5506, a third tine 5508, and a fourth tine 5510 can extend from the base 5502.

As depicted, the first tine 5504 can include a proximal end 5520 and a distal end 5522. Moreover, the first tine 5504 can include an inner surface 5524 and an outer surface 5526. In a particular embodiment, the inner surface 5524 of the first tine 5504 can be relatively longer than the outer surface 5526 of the first tine 5504. As such, the distal end 5522 of the first tine 5504 can be angled from the inner surface 5524 to the outer surface 5526 and this angled portion can face outward relative to the surgical staple 5500.

The second tine 5506 can include a proximal end 5530 and a distal end 5532. Moreover, the second tine 5506 can include an inner surface 5534 and an outer surface 5536. In a particular embodiment, the inner surface 5534 of the second tine 5506 can be relatively longer than the outer surface 5536 of the second tine 5506. As such, the distal end 5532 of the second tine 5506 can be angled from the inner surface 5534 to the outer surface 5536 and this angled portion can face outward relative to the surgical staple 5500.

FIG. 55 also indicates that the base 5502 of the surgical staple 5500 can be formed with a hole 5560. In a particular embodiment, the hole 5560 can facilitate retrieval and insertion of the surgical staple 5500.

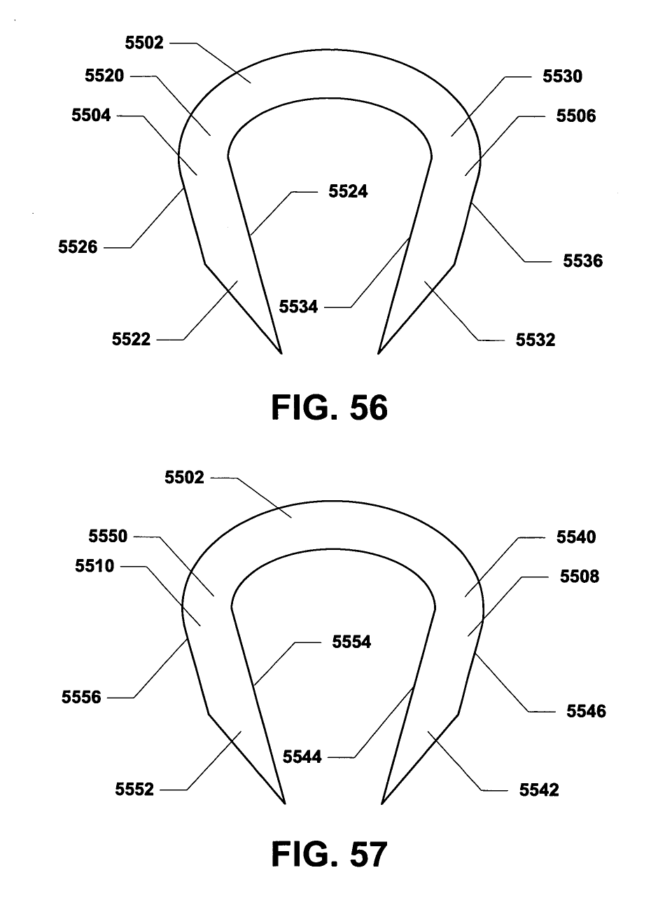

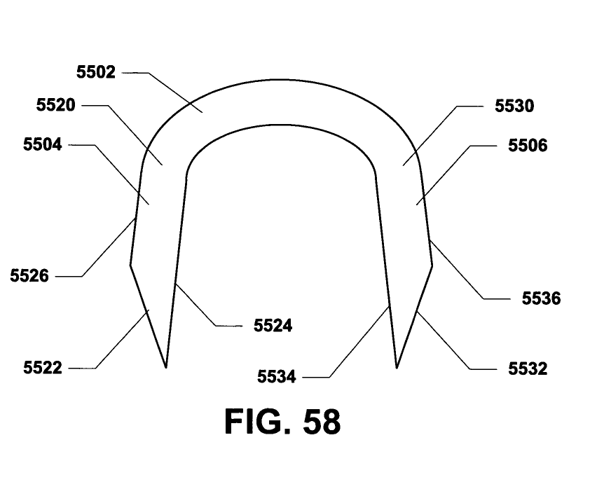

The surgical staple 5500 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. The surgical staple 5500 can be moved between a closed configuration, shown in FIG. 56 and FIG. 57, and an open configuration, shown in FIG. 58. In a particular embodiment, the surgical staple 5500 can be moved from the closed configuration to the open configuration by applying heat to the surgical staple 5500.

Further, in a particular embodiment, the first tine 5504, the second tine 5506, the third tine 5508, the fourth tine 5510, or a combination thereof can include one or more of the enlarged portions described herein.

### Description of a Second Embodiment of a Four-Tine Surgical Staple

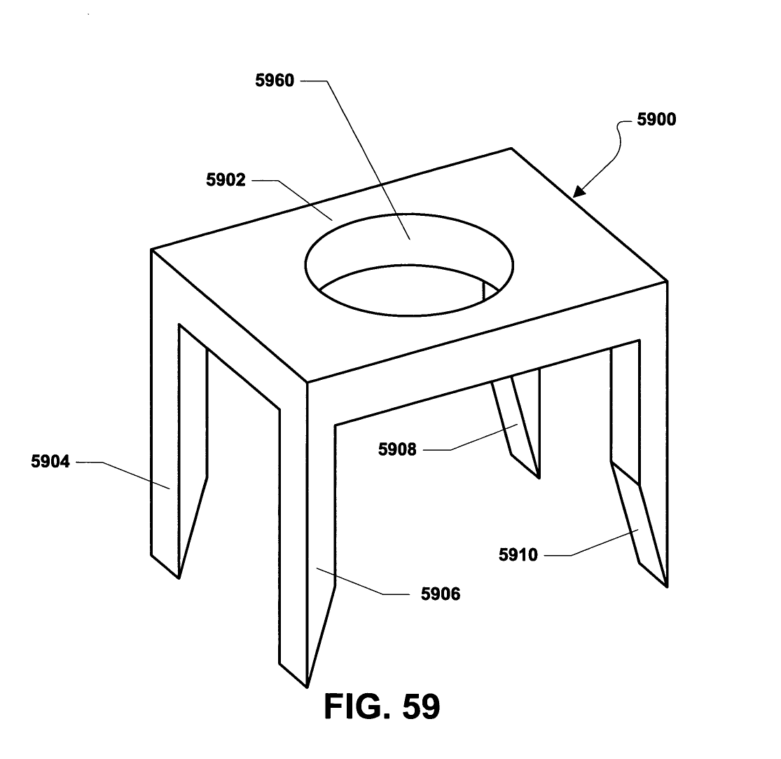

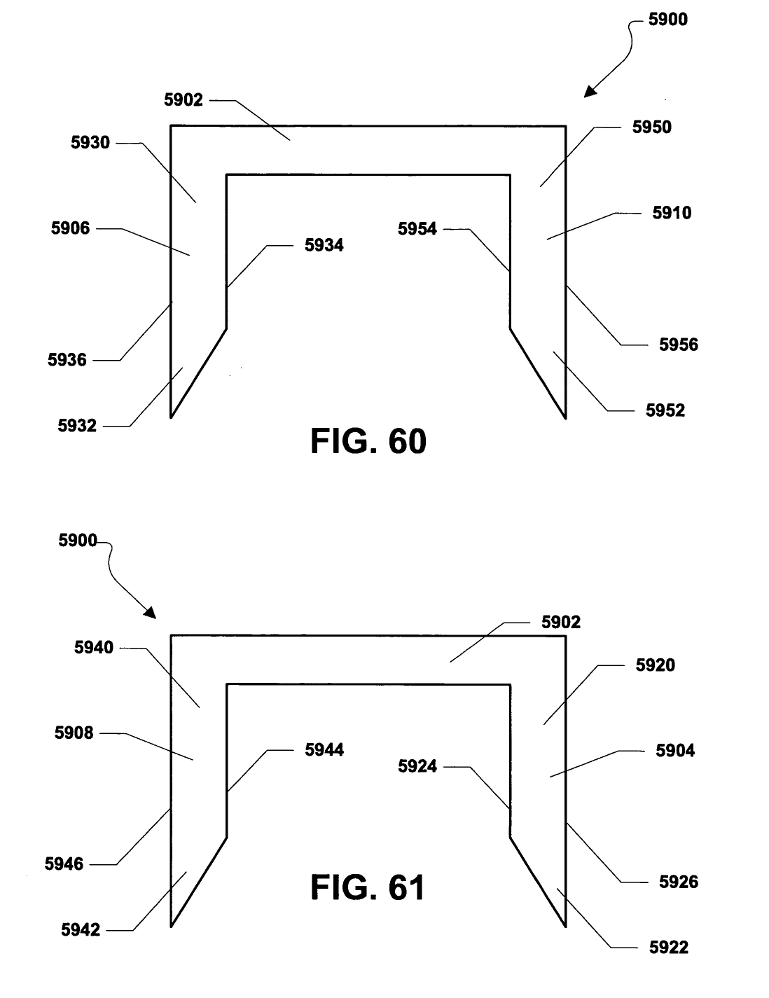

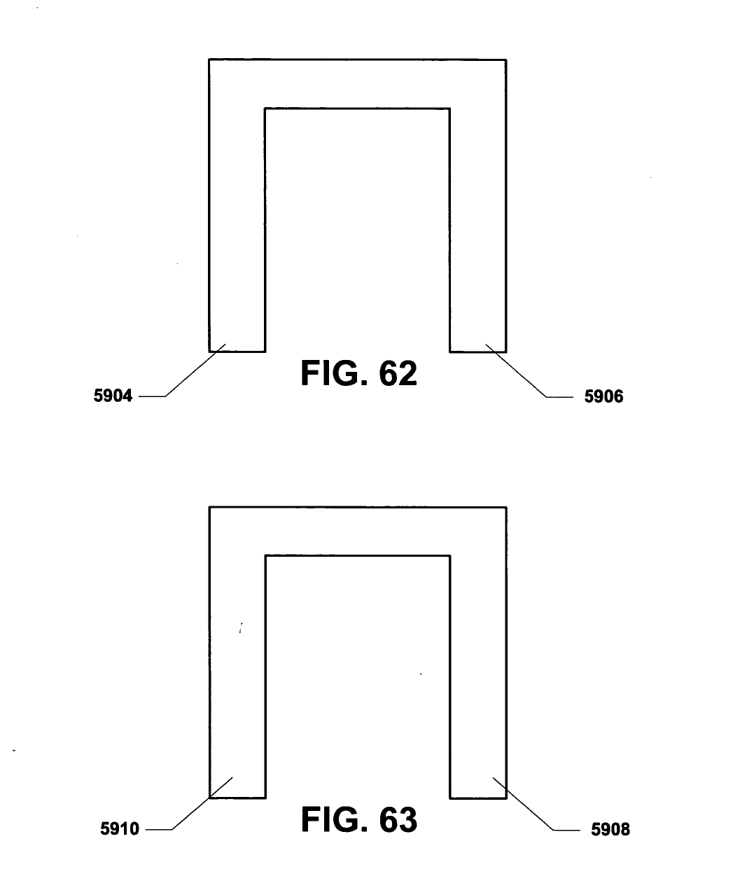

Referring initially to FIG. 59 through FIG. 65, a second embodiment of a four-tine surgical staple is shown and is generally designated 5900. As shown, the four-tine surgical staple 5900 can include a base 5902. A first tine 5904, a second tine 5906, a third tine 5908, and a fourth tine 5910 can extend from the base 5902. For example, the first tine 5904 and the second tine 5906 can extend from one end of the base 5902 and the third tine 5908 and the fourth tine 5910 can extend from the other end of the base 5902. In a particular embodiment, the tines 5904, 5906, 5908, 5910 can extend substantially perpendicular from the base 5902.

As depicted, the first tine 5904 can include a proximal end 5920 and a distal end 5922. Moreover, the first tine 5904 can include an inner surface 5924 and an outer surface 5926. In a particular embodiment, the inner surface 5924 of the first tine 5904 can be relatively shorter than the outer surface 5926 of the first tine 5904. As such, the distal end 5922 of the first tine 5904 can be angled from the inner surface 5924 to the outer surface 5926 and this angled portion can face inward relative to the surgical staple 5900.

The second tine 5906 can include a proximal end 5930 and a distal end 5932. Moreover, the second tine 5906 can include an inner surface 5934 and an outer surface 5936. In a particular embodiment, the inner surface 5934 of the second tine 5906 can be relatively shorter than the outer surface 5936 of the second tine 5906. As such, the distal end 5932 of the second tine 5906 can be angled from the inner surface 5934 to the outer surface 5936 and this angled portion can face inward relative to the surgical staple 5900.

The third tine 5908 can include a proximal end 5940 and a distal end 5942. Moreover, the third tine 5908 can include an inner surface 5944 and an outer surface 5946. In a particular embodiment, the inner surface 5944 of the third tine 5908 can be relatively shorter than the outer surface 5946 of the third tine 5908. As such, the distal end 5942 of the third tine 5908 can be angled from the inner surface 5944 to the outer surface 5946 and this angled portion can face inward relative to the surgical staple 5900.

The fourth tine 5910 can include a proximal end 5950 and a distal end 5952. Moreover, the fourth tine 5910 can include an inner surface 5954 and an outer surface 5956. In a particular embodiment, the inner surface 5954 of the fourth tine 5910 can be relatively shorter than the outer surface 5956 of the fourth tine 5910. As such, the distal end 5952 of the fourth tine 5910 can be angled from the inner surface 5954 to the outer surface 5956 and this angled portion can face inward relative to the surgical staple 5900.

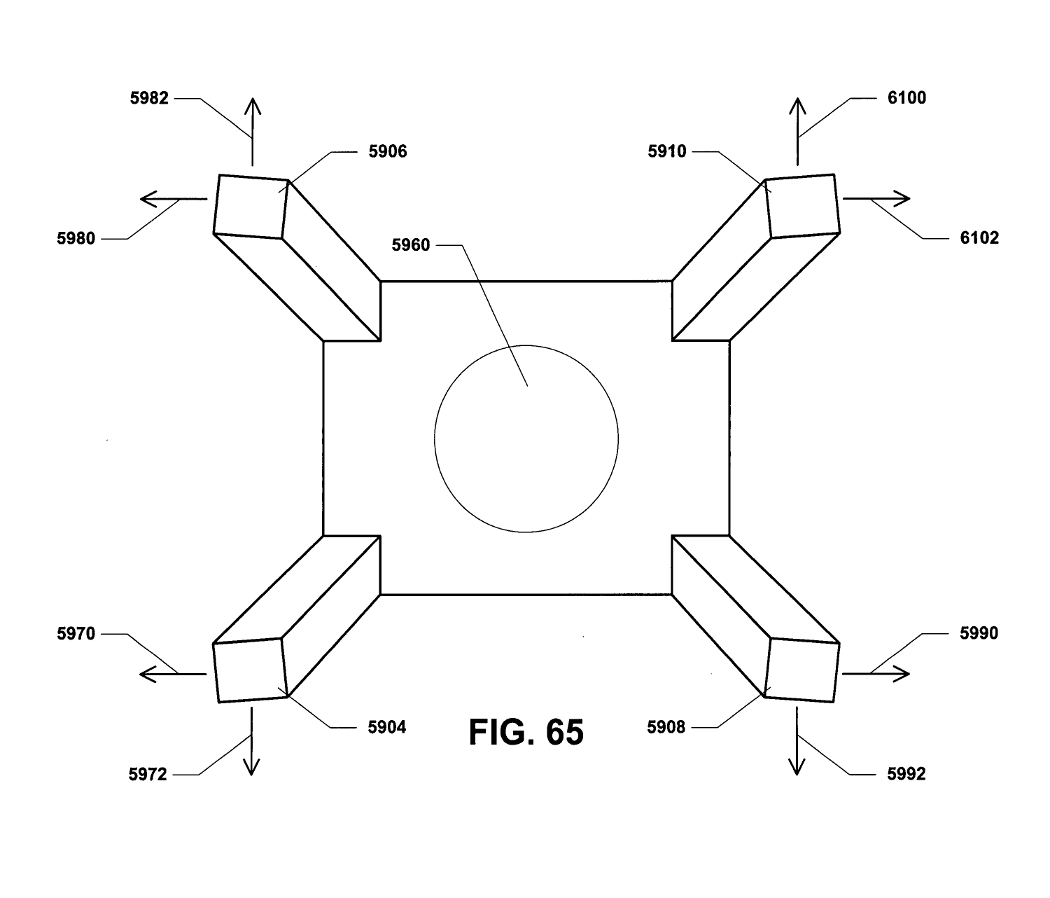

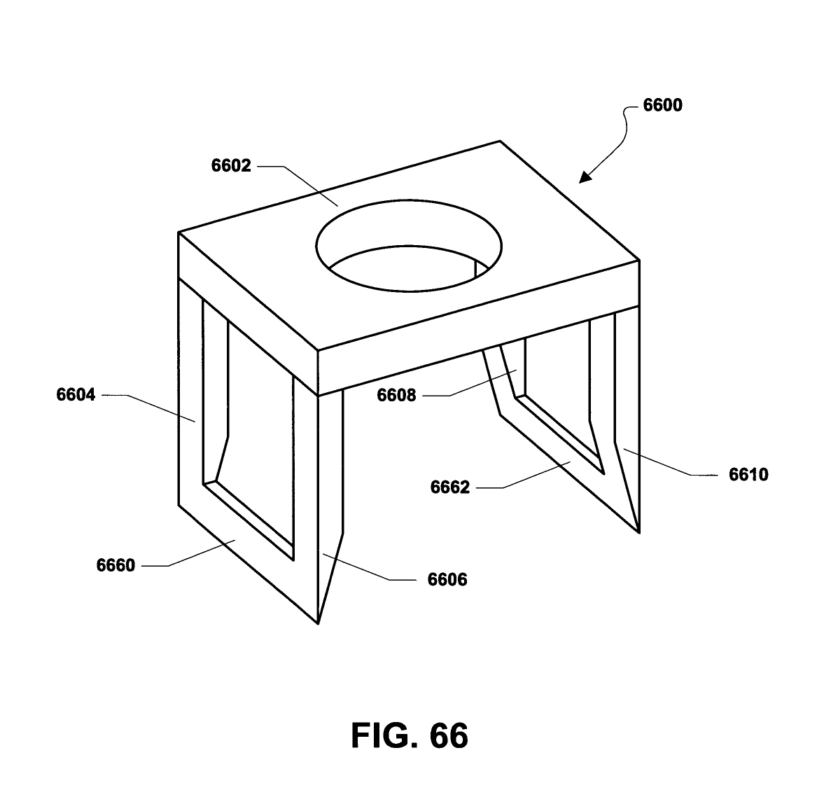

FIG. 59 and FIG. 65 indicate that the base 5902 of the surgical staple 5900 can be generally rectangular. Further, the base 5902 of the surgical staple 5900 can be formed with a hole 5960. In a particular embodiment, the hole 5950 can facilitate retrieval and insertion of the surgical staple 5900.

The surgical staple 5900 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol.

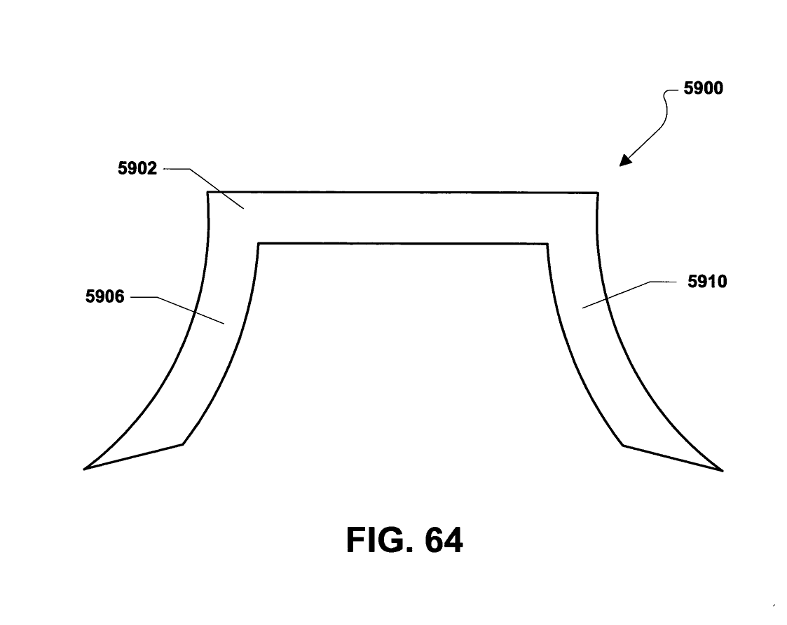

The surgical staple 5900 can be moved between a closed configuration, shown in FIG. 59 through FIG. 63, and an open configuration, shown in FIG. 64 and FIG. 65. In the closed configuration, the tines 5904, 5906, 5908, 5910 of the surgical staple 5900 can be substantially straight and substantially perpendicular to the base 5902 of the surgical staple 5900.

In the open configuration, the tines 5904, 5906, 5908, 5910 of the surgical staple 5900 can be bent outward relative to the base 5902 of the surgical staple 5900. In particular, the first tine 5904 can bend longitudinally with respect to the base 5902, as indicated by arrow 5970, and laterally with respect to the base 5902, as indicated by arrow 5972. Further, the second tine 5906 can bend longitudinally with respect to the base 5902, as indicated by arrow 5980, and laterally with respect to the base 5902, as indicated by arrow 5982. The third tine 5908 can bend longitudinally with respect to the base 5902, as indicated by arrow 5990, and laterally with respect to the base 5902, as indicated by arrow 5992. Also, the fourth tine 5910 can bend longitudinally with respect to the base 5902, as indicated by arrow 6100, and laterally with respect to the base 5902, as indicated by arrow 6102.

Accordingly, the first tine 5904 and the second tine 5906 can bend outward relative to the third tine 5908 and the fourth tine 5910. Moreover, the first tine 5904 and the second tine 5906 can be bend outward relative to each other and the third tine 5908 and the fourth tine 5910 can bend outward relative to each other.

In a particular embodiment, the surgical staple 5900 can be moved from the open configuration to the closed configuration by applying heat to the surgical staple 5900.

Further, in a particular embodiment, the first tine 5904, the second tine 5906, the third tine 5908, the fourth tine 5910, or a combination thereof can include one or more of the enlarged portions described herein.

### Description of a Third Embodiment of a Four-Tine Surgical Staple

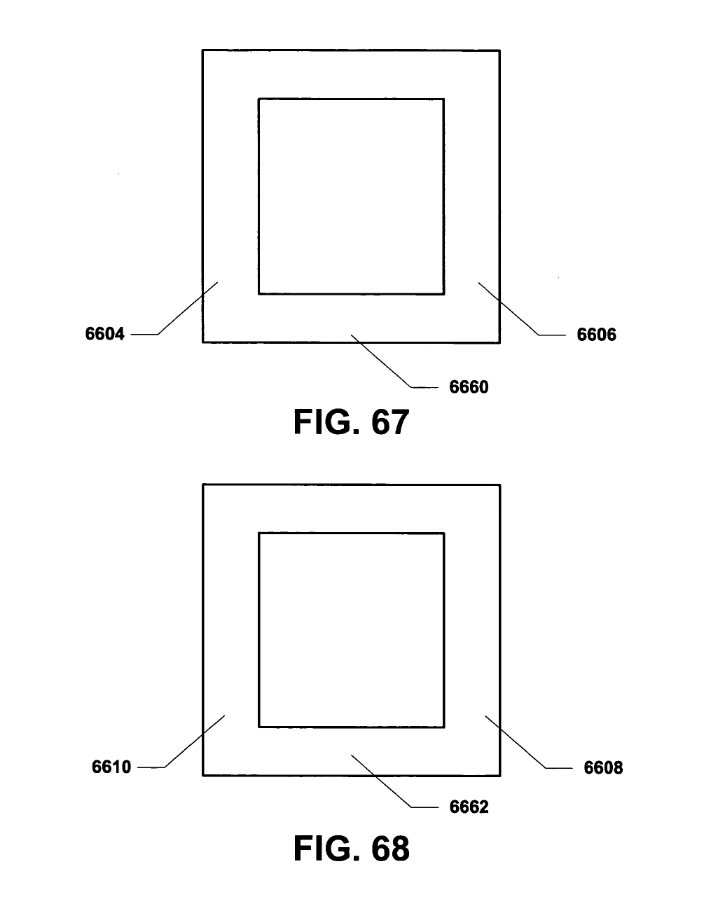

Referring initially to FIG. 66 through FIG. 71, a third embodiment of a four-tine surgical staple is shown and is generally designated 6600. As shown, the four-tine surgical staple 6600 can include a base 6602. A first tine 6604, a second tine 6606, a third tine 6608, and a fourth tine 6610 can extend from the base 6602. For example, the first tine 6604 and the second tine 6606 can extend from one end of the base 6602 and the third tine 6608 and the fourth tine 6610 can extend from the other end of the base 6602. In a particular embodiment, the tines 6604, 6606, 6608, 6610 can extend substantially perpendicular from the base 6602.

As depicted, the first tine 6604 can include a proximal end 6620 and a distal end 6622. Moreover, the first tine 6604 can include an inner surface 6624 and an outer surface 6626. In a particular embodiment, the inner surface 6624 of the first tine 6604 can be relatively shorter than the outer surface 6626 of the first tine 6604. As such, the distal end 6622 of the first tine 6604 can be angled from the inner surface 6624 to the outer surface 6626 and this angled portion can face inward relative to the surgical staple 6600.

The second tine 6606 can include a proximal end 6630 and a distal end 6632. Moreover, the second tine 6606 can include an inner surface 6634 and an outer surface 6636. In a particular embodiment, the inner surface 6634 of the second tine 6606 can be relatively shorter than the outer surface 6636 of the second tine 6606. As such, the distal end 6632 of the second tine 6606 can be angled from the inner surface 6634 to the outer surface 6636 and this angled portion can face inward relative to the surgical staple 6600.

The third tine 6608 can include a proximal end 6640 and a distal end 6642. Moreover, the third tine 6608 can include an inner surface 6644 and an outer surface 6646. In a particular embodiment, the inner surface 6644 of the third tine 6608 can be relatively shorter than the outer surface 6646 of the third tine 6608. As such, the distal end 6642 of the third tine 6608 can be angled from the inner surface 6644 to the outer surface 6646 and this angled portion can face inward relative to the surgical staple 6600.

The fourth tine 6610 can include a proximal end 6650 and a distal end 6652. Moreover, the fourth tine 6610 can include an inner surface 6654 and an outer surface 6656. In a particular embodiment, the inner surface 6654 of the fourth tine 6610 can be relatively shorter than the outer surface 6656 of the fourth tine 6610. As such, the distal end 6652 of the fourth tine 6610 can be angled from the inner surface 6654 to the outer surface 6656 and this angled portion can face inward relative to the surgical staple 6600.

In a particular embodiment, a first arm 6660 can connect the distal end 6622 of the first tine 6604 to the distal end 6632 of the second tine 6606. Further, a second arm 6662 can connect the distal end 6642 of the third tine 6608 to the distal end 6652 of the fourth tine 6610.

FIG. 66 indicate that the base 6602 of the surgical staple 6600 can be generally rectangular. Further, the base 6602 of the surgical staple 6600 can be formed with a hole 6650. In a particular embodiment, the hole 6650 can facilitate retrieval and insertion of the surgical staple 6600.

The surgical staple 6600 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol.

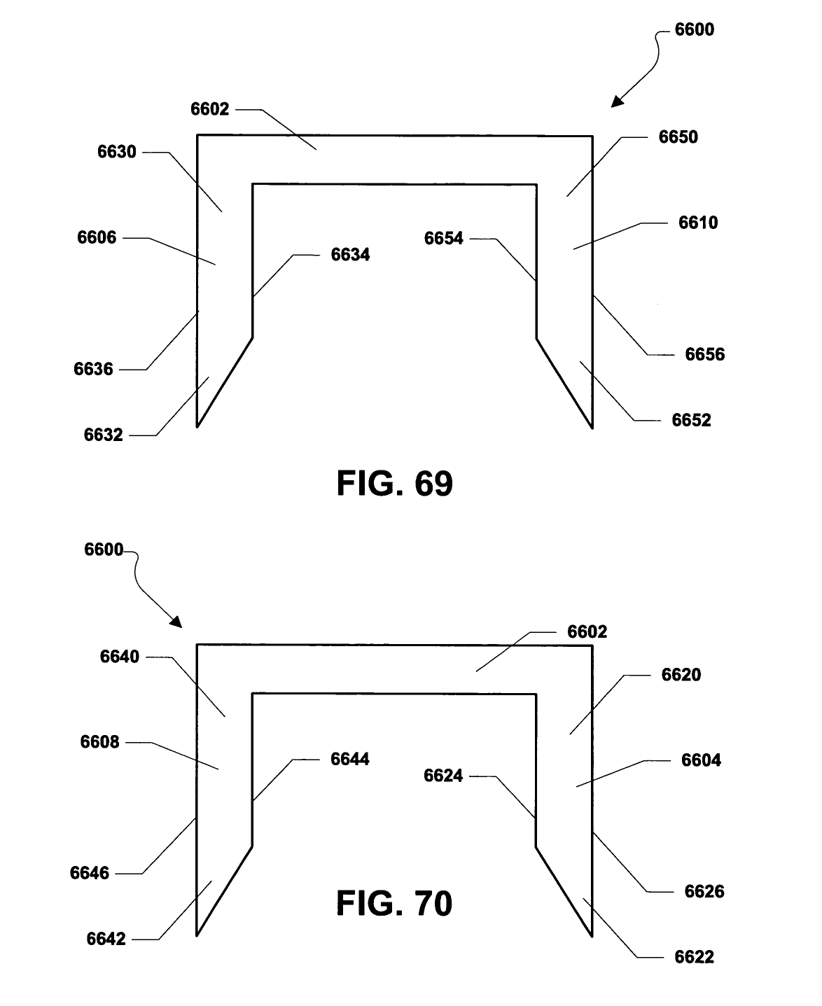

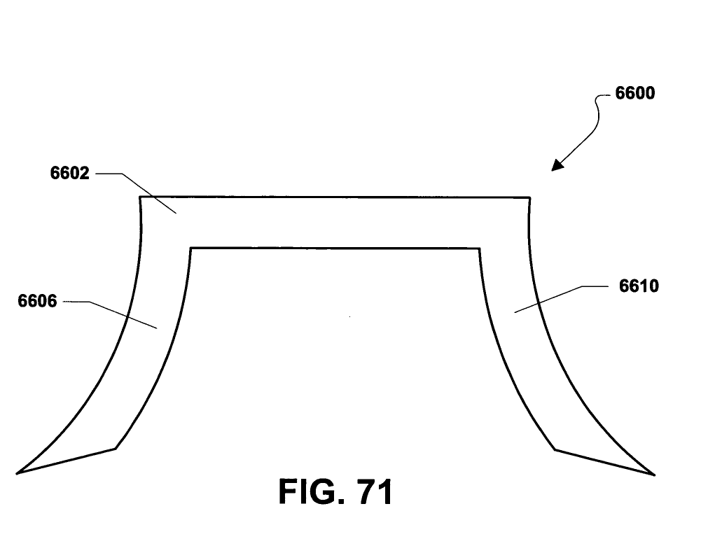

The surgical staple 6600 can be moved between a closed configuration, shown in FIG. 69 through FIG. 70, and an open configuration, shown in FIG. 71. In the closed configuration, the tines 6604, 6606, 6608, 6610 of the surgical staple 6600 can be substantially straight and substantially perpendicular to the base 6602 of the surgical staple 6600.

In the open configuration, the tines 6604, 6606, 6608, 6610 of the surgical staple 6600 can be bent outward relative to the base 6602 of the surgical staple 6600. In particular, the first tine 6604 and the second tine 6606 can bend outward relative to the third tine 6608 and the fourth tine 6610.

In a particular embodiment, the surgical staple 6600 can be moved from the open configuration to the closed configuration by applying heat to the surgical staple 6600.

Further, in a particular embodiment, the first tine 6604, the second tine 6606, the third tine 6608, the fourth tine 6610, or a combination thereof can include one or more of the enlarged portions described herein.

### Conclusion

With the configuration of structure described above, the surgical staple provides a device that can be used to close a wound, e.g., a flesh wound. Further, the surgical staple provides a device that can be used to repair a damaged bone. Also, the surgical staple can be used to treat a spinal deformity. A surgical staple according to one or more of the embodiments herein can include one or more of the various features described herein. Further, a surgical staple according to one or more of the embodiments herein can include any combination of the various features described herein.

In one or more of the embodiments described herein, the tines are angled inward or outward relative to the surgical staple. The angled tines can assist in opening or closing the surgical staple as the surgical staple is inserted in flesh or bone. Further, the angled tines can assist in moving the surgical staple in the direction of a memory shape associated with the surgical staple.

The above-disclosed subject matter is to be considered illustrative, and not restrictive, and the appended claims are intended to cover all such modifications, enhancements, and other embodiments that fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

1. A surgical staple, comprising:
a base; and
at least one tine extending from the base, wherein the at least one tine includes an enlarged portion to provide a greater surface area to engage tissue when installed in a patient.

2. The surgical staple of claim 1, wherein the distal end of the at least one tine includes a necked portion leading to the enlarged portion.

3-6. (canceled)

7. The surgical staple of claim 1, wherein the enlarged portion comprises:
a first surface substantially perpendicular to a longitudinal axis of the surgical staple;
a second surface substantially perpendicular to the longitudinal axis and spaced from the first surface; and
a third surface connecting the first surface and the second surface.

8. The surgical staple of claim 7, wherein the third surface is substantially parallel to the longitudinal axis.

9. The surgical staple of claim 7, wherein the third surface is angled with respect to the longitudinal axis.

10. The surgical staple of claim 1, wherein the enlarged portion comprises:
a first surface substantially perpendicular to a longitudinal axis of the surgical staple;
a second surface connected to the first surface, wherein the second surface is angled with respect to a longitudinal axis of the surgical staple.

11. The surgical staple of claim 1, wherein the at least one tine includes at least one barb extending therefrom.

12. The surgical staple of claim 1, wherein the at least one tine includes at least one ring circumscribing the tine, wherein the at least one ring extends outward from the tine.

13. The surgical staple of claim 1, wherein the at least one tine comprises a first tine and a second tine.

14. The surgical staple of claim 13, wherein the surgical staple is movable between a closed configuration and an open configuration.

15. The surgical staple of claim 13, wherein the surgical staple is movable between a straight configuration and a twisted configuration.

16. The surgical staple of claim 15, wherein in the straight configuration a distal end of each tine is substantially parallel to a proximal end of each tine.

17. The surgical staple of claim 16, wherein in the twisted configuration the distal end of each tine is twisted relative to the proximal end of each tine.

18. The surgical staple of claim 17, wherein in the distal end of each tine is twisted ninety degrees (90°) relative to the proximal end of each tine.

19-30. (canceled)

31. The surgical staple of claim 1, wherein the distal end of the at least one tine includes an angled portion and wherein the angled portion of the tine moves the tine inward or outward as the surgical staple is driven into tissue.

32. The surgical staple of claim 31, wherein the angled portion of the at least one time moves the tine toward a memory shape as the surgical staple is driven into tissue.

33. A surgical staple, comprising:
a base;
a first tine extending from the base;
an enlarged portion formed along the first tine;
a second tine extending from the base opposite the first tine; and
an enlarged portion formed along the second tine, wherein the surgical staple is movable between a closed configuration and an open configuration.

34. A surgical staple, comprising:
a base;
a first tine extending from the base;
an enlarged portion formed along the first tine;
a second tine extending from the base opposite the first tine; and
an enlarged portion formed along the second tine, wherein the surgical staple is movable between a straight configuration and a twisted configuration.

35-38. (canceled)

39. A surgical staple, comprising:
a generally trough-shaped base;
a first hollow tine extending from the base; and
a second hollow tine extending from the base opposite the first hollow tine.

40. The surgical staple of claim 39, wherein the first hollow tine is generally cylindrical.

41. The surgical staple of claim 40, wherein the second hollow tine is generally cylindrical.

42. The surgical staple of claim 39, wherein the first hollow tine extends substantially perpendicular from the base.

43. The surgical staple of claim 42, wherein the second hollow tine extends substantially perpendicular from the base.

44. The surgical staple of claim 39, wherein the surgical staple is movable between a closed configuration in which each hollow tine is substantially straight and an open configuration in which each hollow tine is bent.

45. The surgical staple of any of the preceding claims, wherein at least a portion of the staple comprises a shape memory material.

46. The surgical staple of claim 45, wherein the shape memory material is nitinol.
